(19) United States
(12) Patent Application Publication
(10) Pub. No.: US 2022/0325250 A1
(43) Pub. Date: Oct. 13, 2022

(54) METHOD FOR OBTAINING EFFICIENT COMPOSITIONS COMPRISING VIRAL VECTORS FOR VACCINATION OR GENE THERAPY

(71) Applicant: Leukocare AG, Martinsried (DE)

(72) Inventors: Eva Reinauer, Eurasburg (DE); Kristina Kemter, Garching (DE); Jens Altrichter, Ostseebad Dierhagen (DE)

(73) Assignee: Leukocare AG, Martinsried (DE)

(21) Appl. No.: 17/635,109

(22) PCT Filed: Aug. 14, 2020
(86) PCT No.: PCT/EP2020/072832

§ 371 Date: Feb. 14, 2022

(30) Foreign Application Priority Data
Aug. 14, 2019 (EP) 19191690.7

(51) Int. Cl. C12N 7/00

(52) U.S. Cl. CPC C12N 7/00; C12N 2750/14143; C12N 2750/14151

(57) ABSTRACT

The present invention relates to a method for preparing a composition comprising a viral vector, the method comprising the steps of a) providing viral vectors, (b) providing a solution comprising at least one sugar and at least three different excipients selected from hydrophilic and amphiphilic excipients, wherein the excipients are characterized by polar, aliphatic, aromatic, negatively charged, and/or positively charged functional groups, and wherein the solution is free or substantially free of Mg2+ or of any divalent cations and/or salts thereof; and (c) mixing the replication deficient viral vectors of step (a) with the solution of step (b). Furthermore, the invention relates to a composition obtained or obtainable by the method of the invention, and to a composition comprising a viral vector and the solution of step (b).

The present invention relates to a method for preparing a composition comprising a viral vector, the method comprising the steps of a) providing viral vectors, (b) providing a solution comprising at least one sugar and at least three different excipients selected from hydrophilic and amphiphilic excipients, wherein the excipients are characterized by polar, aliphatic, aromatic, negatively charged, and/or positively charged functional groups, and wherein the solution is free or substantially free of Mg²⁺ or of any divalent cations and/or salts thereof; and (c) mixing the replication deficient viral vectors of step (a) with the solution of step (b). Furthermore, the invention relates to a composition obtained or obtainable by the method of the invention, and to a composition comprising a viral vector and the solution of step (b).

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

Replication-deficient recombinant viral vectors represent a rapidly growing field of vaccine development and gene therapy. When intended for use in vaccination, viral vectors and virus like particles (VLPs) offer a series of advantages over traditional vaccines. In addition to inducing exceptional antibody responses, they also elicit cytotoxic T lymphocytes (CTL) that are crucial for the control of intracellular pathogens and cancer, a feature not observed by protein-based vaccines (Rollier C S et al., 2011, Curr Opin Immunol. 23(3):377-382. doi:10.1016/j.coi.2011.03.006). Many viral species have been evaluated as recombinant vectors for vaccines, including retrovirus, lentivirus, vaccinia virus (e.g. modified vaccinia Ankara virus; MVA), adenovirus, adeno-associated virus, cytomegalovirus, Sendai virus, measles virus and vesicular stomatitis virus (VSV). However, the most widely evaluated vectors to date are adenovirus type 5 and members of the poxvirus family (Rollier C S et al., 2011, and Ura T et al. 2014, Vaccines (Basel). July 29; 2(3):624-41).

A drawback associated with viral vectors, in particular upon manufacturing, storage and distribution, is that they are complex supra-molecular ensembles of macromolecules which are prone to a variety of chemical and physical degradation pathways [Vrdoljak A et al. 2012]. Thus, a major challenge in this field is the reduction (avoidance) of cross-linking and vector particle interaction of neighboring virus particles that is typically caused over a broad range of concentrations by various mechanisms at different stages of production, storage and application. This intrinsic tendency of viral vectors for particle agglomeration of different shapes and sizes within a composition leads to inhomogeneous size distribution of the viral particles and an associated increase in polydispersity. Ultimately, these effects result in a significant loss of therapeutic efficacy, and can even lead to adverse effects at the injection site, most likely due to the increased viscosity observed as a result of said particle agglomeration. Furthermore, aggregation of the viral vectors is also considered to influence biodistribution after administration and, similar to protein pharmaceuticals, aggregation of viral vectors may increase undesired immunogenicity by targeting the vector to antigen presenting cells, thereby inducing or enhancing undesired immune responses to the surface proteins or protein capsids and transgenic products. As high polydispersity is associated with high viscosity, compositions that do not show such unappreciated polydispersity are expected to also lead to better syringeability and injectability. Thus, improved viral vector-based vaccines with low polydispersity and having a more suitable ratio between vector particle distribution and functional efficacy would be highly desired.

Similar considerations apply when viral vectors are intended for use as gene transfer therapeutics. Viral vectors have emerged as safe and effective delivery vehicles for clinical gene therapy, as shown in a series of clinical studies, especially for monogenic recessive disorders, but also for some idiopathic. These clinical studies were conducted on the basis of vectors that combine low genotoxicity and immunogenicity with highly efficient delivery, including vehicles based on adeno-associated virus and lentivirus, which are increasingly enabling clinical success. Important examples for clinical treatment strategies based on viral vectors include, e.g., stem cell therapy, mucoviscidosis, haemophilia, inherited retinopathy or cystic fibrosis. (Collins M, Thrasher A, Gene therapy: progress and predictions. Proc Biol Sci. 2015; 282). Typically, the viral vectors employed in such gene transfer therapeutics include retrovirus, adenovirus, adeno-associated virus (AAV) and herpes simplex virus.

Also with regard to gene transfer therapeutics, the avoidance of unappreciated polydispersity for obtaining a more suitable ratio between vector particle distribution and functional efficacy of gene transfer vectors is essential for efficient host cell infection and subsequent gene expression. In particular, the in vivo administration of gene therapeutic viral vectors to certain sites, such as the central nervous system, is expected to require small volumes of highly concentrated viral vectors, a feature for which the maximum achievable dose may be limited by the intrinsic property of low vector solubility. Thus, at present, there are still substantial delivery challenges that have to be overcome to extend the success achieved so far to a broad variety of diseases; these challenges include developing techniques to evade pre-existing immunity, to ensure more efficient transduction of therapeutically relevant cell types, to target delivery, and to ensure genomic maintenance.

Formulation development for virus-based viral vector compositions for vaccines or gene-transfer therapeutics is rather difficult, mainly due to their complex molecular structure. Thus, formulation development for viral vector based pharmaceutical compositions is a relatively recent area of investigation and only a few studies and patent applications have been reported describing systematic efforts to optimize viral vector formulations and stability. An important aspect of vector stability is solubility during vector purification, preparation and storage. Ultimately, maintenance of transfection/infection properties of the viral vectors is a final goal of stabilizing the viral vectors.

Generally, many virus formulations known in the art employ divalent ions such as MgCl₂ as a stabilizing agent.

WO 00/32233 A2 discloses AAV virus formulations comprising dihydric or polyhydric alcohols such as polyethylene glycol, propylene glycol and sorbitol as stabilizing excipients. The use of mixtures of different amino acids is not disclosed.

WO 2005/118792 A1 discloses AAV virus formulations with high ionic strength to prevent aggregation. Preferred excipients are multivalent ions such as magnesium ions.

WO 01/66137 A1 discloses liquid adenovirus formulations showing improved stability when stored in about the 2-8° C. for 28 days. The formulations comprise a buffer, a sugar, a salt, a divalent cation, such as MgCl₂, a non-ionic detergent, as well as a free radical scavenger and/or a chelating agent to inhibit free radical oxidation.

WO 2018/050872 A1 discloses liquid adenovirus formulations comprising different mixtures of amino acids, saccharose and MgCl₂. The formulations were stored over a maximum storage time of 84 days at 25° C. and 35 days at 37° C.

In view of the art it is generally desirable to further increase the stability of viral vector formulations, especially with regard to infectivity, to provide formulations that can be stored over extended periods of time.

This need is addressed by the provision of the embodiments characterized in the claims.

Accordingly, the present invention relates to a method for preparing a composition comprising a viral vector, the method comprising the steps: (a) providing viral vectors; (b) providing a solution comprising at least one sugar and at least three different excipients selected from hydrophilic and amphiphilic excipients, wherein the excipients are characterized by polar, aliphatic, aromatic, negatively charged, and/or positively charged functional groups, and wherein the solution is free or substantially free of Mg²⁺ and/or salts thereof; and (c) mixing the replication deficient viral vectors of step (a) with the solution of step (b).

In a highly preferred embodiment, the at least three different excipients comprise at least one amino acids or the at least three different excipients are three different amino acids.

In a highly preferred embodiment, the solution is free or substantially free of any divalent cations and/or salts thereof.

The term “free or substantially free of Mg²⁺” or “free or substantially free of divalent cations”, in accordance with the present invention, refers to a solution or solid composition devoid of or substantially devoid of Mg²⁺, or any types of divalent cations either in form of solubilized ions or salts of divalent cations, respectively. A solution or composition is considered substantially free of Mg²⁺ or any divalent cations even if it comprises small amounts of Mg²⁺ or any divalent cations that typically result from inevitable impurities in the other constituents of the solution or solid composition. The solution is considered to be substantially free of Mg²⁺ or any divalent cation if it contains less than 100 μM of Mg²⁺ or a divalent cation, more preferably less than 10 μM and most preferably less than 1 μM. The solid composition is considered to be substantially free of Mg²⁺ or any divalent cation if it contains less than 0.01% (w/v) of a Mg²⁺ or divalent cation, more preferably less than 0.001% (w/v) and most preferably less than 0.0001% (w/v).

For example, the solution is free or substantially free of Ca²⁺, Mn²⁺, Cu²⁺, Zn²⁺, and/or Ni²⁺ and salts thereof. Especially, the solution is free or substantially free of MgCL₂.

Viral vectors are commonly used to deliver genetic material into cells in vivo or in vitro. Viruses may efficiently transport their genomes inside the host cells. Virus-like particles resemble viruses, are non-infectious and do not contain viral genetic material. The expression of viral structural proteins, such as envelope or capsid, can result in the self-assembly of virus like particles (VLPs). VLPs derived from the Hepatitis B virus may be composed of the HBV surface antigen (HBsAg) (Hyakumura M. et al. J. Virol. 89:11312-22, 2015) or from HBV core (Sominskaya I. et al. PLos One 8:e75938). VLPs have been produced from components of various virus families including Parvoviridae (e.g. adeno-associated virus), Retroviridae (e.g. HIV), Flaviviridae (e.g. Hepatitis C virus) and bacteriophages (e.g. Qβ, AP205). VLPs can be produced in different cell culture systems including bacterial, mammalian, insect, yeast and plant cell lines.

The term “viral vector-based composition” as used herein, relates to a composition that comprises at least a viral vector.

The term “viral vector”, in accordance with the present invention, relates to a carrier, i.e. a “vector” that is derived from a virus. “Viral vectors” in accordance with the present invention include vectors derived from naturally occurring or modified viruses, as well as virus like particles (VLPs). “Viral vector” may be viruses derived from naturally occurring viruses by genetic modification. The term “viral vectors” may relate to multiple individual vector entities of the same vector type or multiple individual vector entities of different vector types.

In general, the starting materials for the development of viral vectors are live viruses. Thus, certain requirements such as safety and specificity need to be fulfilled in order to ensure their suitability for use in animals or in human patients. One important aspect is the avoidance of uncontrolled replication of the viral vector. This is usually achieved by the deletion of a part of the viral genome critical for viral replication. Such a virus can infect target cells without subsequent production of new virions. Moreover, the viral vector should have no effect or only a minimal effect on the physiology of the target cell and rearrangement of the viral vector genome should not occur. Such viral vectors derived from naturally occurring or modified viruses are well known in the art and have been described, e.g. in the Review of Lukashev A N and Zamyatnin A A “Viral Vectors for Gene Therapy: Current State and Clinical Perspectives”. Front Mol Neurosci. 2016; 9:56 as well as in the Review of Stoica L and Sena-Esteves M “Adeno Associated Viral Vector Delivered RNAi for Gene Therapy of SOD1 Amyotrophic Lateral Sclerosis”, Front Mol Neurosci. 2016 Aug. 2; 9:56.

Also vectors derived from virus like particles are well known in the art and have been described, e.g. in Tegerstedt et al. (Tegerstedt et al. (2005), Murine polyomavirus virus-like particles (VLPs) as vectors for gene and immune therapy and vaccines against viral infections and cancer. Anticancer Res. 25(4):2601-8.). One major advantage of VLPs is that they are not associated with any risk of reassembly as is possible when live attenuated viruses are used as viral vectors and, as such, they represent “replication-deficient viral vectors” in accordance with the present invention. VLP production has the additional advantage that it can be started earlier than production of traditional vaccines once the genetic sequence of a particular virus strain of interest has become available. VLPs contain repetitive high density displays of viral surface proteins which present conformational viral epitopes that can elicit strong T cell and B cell immune responses. VLPs have already been used to develop FDA approved vaccines for Hepatitis B and human papillomavirus and, moreover, VLPs have been used to develop a preclinical vaccine against chikungunya virus. Evidence further suggests that VLP vaccines against influenza virus might be superior in protection against flu viruses over other vaccines. In early clinical trials, VLP vaccines for influenza appeared to provide complete protection against both the Influenza A virus subtype H5N1 and the 1918 flu as reviewed by Quan F S et al., “Progress in developing virus-like particle influenza vaccines”. Expert Rev Vaccines. 2016 May 5:1-13.

Highly purified and homogenous VLPs can be formulated as so-called “lipoparticles”, which contain high concentrations of a conformationally intact membrane protein of interest. Integral membrane proteins are involved in diverse biological functions and are targeted by nearly 50% of existing therapeutic drugs. However, because of their hydrophobic domains, membrane proteins are difficult to manipulate outside of living cells. Lipoparticles can incorporate a wide variety of structurally intact membrane proteins, including G protein-coupled receptors (GPCR)s, ion channels, and viral envelopes. Lipoparticles may be used as platform for numerous applications including antibody screening, production of immunogens, and ligand binding assays.

Virus-like particles can also be used as drug delivery vectors (Zdanowicz M and Chroboczek J, Virus-like particles as drug delivery vectors. Acta Biochim Pol. 2016; 63(3):469-473.).

The presence of viral structural proteins, for example, structural proteins in the envelope or in the capsid, can result in the self-assembly of VLPs. In general, VLPs can be produced in a variety of cell culture systems including mammalian cell lines, insect cell lines, yeast, and plant cells and VLPs have been produced from different virus families including parvoviridae (e.g. adeno-associated virus), retroviridae (e.g. HIV), and flaviviridae (e.g. Hepatitis C virus). For example, VLPs derived from the Hepatitis B virus and composed of the small HBV-derived surface antigen (HBsAg) have been described by Sominskaya I et al. (Sominskaya I et al. Construction and immunological evaluation of multivalent hepatitis B virus (HBV) core virus-like particles carrying HBV and HCV epitopes. Clin Vaccine Immunol. 2010 June; 17:1027-33).

In accordance with the present invention, the term “viral vectors” includes, without being limiting, (i) viral vectors represented by one particular type of viral vector, or (ii) viral vector mixtures of different molecular types of viral vectors.

The composition may, optionally, comprise further molecules capable of altering the characteristics of the viral vector(s). For example, such further molecules can serve to stabilize, modulate and/or enhance the function of the viral vector(s). The compositions comprising viral vectors prepared by the method of the present invention may be in solid or liquid form and may be, inter alia, in the form of (a) powder(s), (a) tablet(s) or (a) solution(s).

In an embodiment of the invention, the virus comprising composition prepared in accordance with the present invention may be further characterized in that the particles comprised in the composition have a particle size distribution with a polydispersity index (PDI) of less than 0.5.

The term “particle(s)”, as used herein, relates to the viral vector(s) that represent the main, active ingredient of the composition prepared in accordance with the invention. The term “particle size distribution”, in accordance with the present invention, refers to the relative amount of particles present according to size. Typically, the relative amount is determined by mass.

In accordance with the present invention, the particle size distribution is expressed in terms of the polydispersity index (PDI). Polydispersity and the polydispersity index are parameters measured by Dynamic Light Scattering (DLS) and characterize a dispersion or solution in addition to the typically determined main parameters, i.e. particle size and hydrodynamic diameter of particles. DLS measures time-dependent fluctuations in the scattering intensity arising from particles, such as e.g. viral particles or proteins undergoing random Brownian motions (diffusion). A monochromatic light beam, such as a laser beam, causes a Doppler shift in a solution with particles in Brownian motion when the light hits the moving particles, thereby changing the wavelength (typically red light at 633 nm or near-infrared at 830 nm) of the incoming light—this change is related to the size of the particles. The particles in a liquid move about randomly and their motion is used to determine the size of the particles: small particles are moving quickly resulting in a more rapid intensity fluctuation, whereas large particles are moving slowly, leading to slower intensity fluctuations.

Construction of the time-dependent autocorrelation function from the measured intensity fluctuation and fitting of this correlation curve to an exponential function gives a description of the particle motion in the medium by calculation of the Diffusion coefficient of the Brownian molecular motion. The hydrodynamic diameter of the particles can subsequently be calculated by using the Stokes-Einstein equation. For polydisperse samples, this curve is a sum of exponential decays. The polydispersity index (PDI) is a parameter derived from the cumulant analysis of the DLS measured intensity autocorrelation function originally introduced by D. E. Koppel in The Journal of Chemical Physics 57(11); 1972; pp: 4814-20. In the cumulant analysis, a single exponential fit is applied to the resulting autocorrelation function by the applied DLS software assuming a single-sized population following a Gaussian distribution. The polydispersity index is related to the standard deviation (σ) of the hypothetical Gaussian distribution around the assumed particle size population in the following fashion:

PDI=σ²/Z_{D}²,

where Z_{D} is Z-average size or cumulants mean, the intensity weighted mean hydrodynamic size of the ensemble collection of particle, representing the average of several species in the case of polydisperse samples (Stepto, R F T et al. (2009). “Dispersity in Polymer Science” Pure Appl. Chem. 81 (2): 351-353).

Calculated polydispersity indices are dimensionless parameters representing the width of the particle size distribution in the solution. PDI values between 0.1 to 0.2 correspond to a narrow particle size distribution approximately representing a monodisperse particle size distribution. PDI values around 0.3 suggest an increasing width of the particle size distribution containing an increasing number of different particle populations. Values ranging between 0.5 and 0.7 represent a very broad particle size distribution containing very large particles or aggregates. PDI values greater than 0.7 indicate the sample has a very broad particle size distribution and may contain large particles or aggregates. In other words, the lower the PDI value, the more predominant infective viral particles species are present, i.e. viral particles species with a narrow particle size and without or with only a small amount of aggregates and, accordingly, a higher efficacy of the viral vector composition can be achieved.

According to the invention, determination of PDI based on DSL may for example be performed according to ISO Norm 22412:2017 and/or ISO Norm 13321:1996 E.

In accordance with the present invention, the PDI is less than 0.5. As described above, this PDI indicates a particle size distribution ranging from almost monodisperse to moderate polydisperse, with infective particles as the predominant species and only a minor portion of large particles or agglomerates, or even without any large particles or agglomerates. Preferably, the PDI is less than 0.3, more preferably less than 0.2 and most preferably less than 0.1.

In accordance with the present invention and the applied example 5, the preferred PDI value is less than 0.5 for enveloped viruses, e.g. MVA, and less than 0.3 for non-enveloped viruses, e.g. adenoviruses. Furthermore, it is preferred to maintain the above mentioned PDI values during viral vector processing, manufacturing, and distribution phases.

The method of the present invention comprises in a first step (a) the provision of viral vectors.

In a preferred embodiment, the viral vectors are replication-deficient viral vectors.

Replication-deficient viral vectors are viral vectors that are not capable of replicating to generate new viral particles in host cells. For example, the viral vectors can have lost their replication competence by empirical and rational attenuation processes resulting in a loss of important parts of their genome accompanied by (i) retention of their ability to infect several cell types, and (ii) retention of their immunogenicity. Also VLPs fall under the term “replication-deficient viral vector”, in accordance with the present invention.

Due to the lack of replication competence, replication-deficient viral vectors represent safe and robust mechanism to induce both effector cell mediated and humoral immunity. As a consequence, priming with these vectors can improve the magnitude, quality and durability of such responses, while at the same time providing an increased safety.

Suitable replication-deficient viral vectors for vaccine preparation are well known in the art. For example, Verheust C. et al. (Vaccine 30, 2012) provides a review regarding modified vaccinia Ankara virus (MVA)-based vectors, Rosewell A et al., (J Genet Syndr Gene Ther, 2011) provides a review regarding helper-dependent adenoviral vectors, and Mulder A M et al. (PlosOne 7, 2012) provides a review regarding recombinant VLP-based vaccines. The considerations for choosing a suitable viral vector for vaccine production commonly applied in the art apply mutatis mutandis with regard to choosing a suitable viral vector for vaccine production in accordance with the present invention. Accordingly, viral vectors already available in the art, as well as novel viral vectors, may be employed in the claimed method.

Preferably, the replication-deficient viral vectors are selected from the group consisting of MVA, adenovirus, adeno associated virus, lentivirus, Vesicular stomatitis virus, herpes simplex virus, or measles virus. Most preferably, the replication-deficient viral vector is modified vaccinia Ankara virus (MVA) or adenovirus.

The viral vectors employed in the invention can be freshly prepared, e.g. reconstituted after harvesting from cell cultures, or can be provided as a pre-prepared composition, for example from commercial sources.

In a second step (b), the method comprises the provision of a solution comprising at least one sugar and at least three different excipients selected from hydrophilic and amphiphilic excipients, wherein the excipients are characterized by polar, aliphatic, aromatic, negatively charged, and/or positively charged functional groups.

The solution, in accordance with the present invention, can be an aqueous or a non-aqueous solution. In the context of the present invention, the term “aqueous solution” refers on one hand to water but extends on the other hand also to buffered solutions and hydrophilic solvents miscible with water, thus being able to form a uniform phase. Examples for aqueous solutions include, without being limited, water, methanol, ethanol or higher alcohols as well as mixtures thereof. Non-limiting examples for non-aqueous solvents include dimethylsulfoxide (DMSO), ethylbenzene, and other polar solvents.

The term “comprising”, as used in accordance with the present invention, denotes that further steps and/or components can be included in addition to the specifically recited steps and/or components. However, this term also encompasses that the claimed subject-matter consists of exactly the recited steps and/or components.

Non-limiting examples of further components that can be comprised in the solution according to step (b) of the method of the invention include e.g., water, amino acids, buffers such as phosphate, citrate, succinate, acetic acid, histidine, glycine, arginine and other organic acids or their salts; antioxidants such as ascorbic acid, methionine, tryptophan, cysteine, glutathione, chelating agents such as ethylenediaminetetraacetic acid (EDTA); counterions such as sodium; and/or nonionic surfactants such as polysorbates, poloxamers, or PEG or other solvents. Preferably, the solution does not contain any proteins other than the (viral) proteins that are part of the viral vectors and the above included components in form of a pharmaceutical carrier.

The solution according to step (b) of the method of the invention further comprises at least one sugar. In an embodiment of the invention, the solution comprises an excipient-sugar ratio of at least 1:2 (w/w). A excipient-sugar ration of at least 1:2 refers to a ratio of 1 or more parts of excipient to 2 parts of sugar.

The term “sugar”, as used herein, refers to any types of sugars, i.e. the monosaccharide, disaccharide or oligosaccharide forms of carbohydrates as well as sugar alcohols. Examples of suitable sugars include, without being limiting, trehalose, saccharose, sucrose, glucose, lactose, mannitol, and sorbitol or sugar derivatives such as aminosugars, e.g glucosamine or n-acetyl glucosamine.

The term “at least”, as used herein, refers to the specifically recited amount or number but also to more than the specifically recited amount or number. For example, the term “at least one” encompasses also at least 2, at least 3, at least 4, at least 5, at least 6, at least 7, at least 8, at least 9, at least 10, such as at least 20, at least 30, at least 40, at least 50 and so on. Furthermore, this term also encompasses exactly 1, exactly 2, exactly 3, exactly 4, exactly 5, exactly 6, exactly 7, exactly 8, exactly 9, exactly 10, exactly 20, exactly 30, exactly 40, exactly 50 and so on.

It will further be appreciated that the term “one sugar” means one type of sugar and does not limit the number of molecules of this particular type of sugar to one. Further, in those cases where more than one sugar is comprised, such as e.g. two sugars, two different types of sugar envisaged. Preferably, the solution comprises exactly one type of sugar, preferably trehalose.

Preferred amounts of sugars to be comprised in the solution according to the invention are between 0.1 mg/ml to 200 mg/ml sugar, more preferably between 10 mg/ml to 180 mg/ml sugar, even more preferably between 20 mg/ml to 160 mg/ml sugar and most preferably the amount is about 80 mg/ml sugar. Where a mixture of different types of sugars is employed, these preferred amounts refer to the sum of all sugars in the solution.

The term “about”, as used herein, encompasses the explicitly recited values as well as small deviations therefrom. In other words, an amount of sugar of “about 80 mg/ml” includes, but does not have to be exactly the recited amount of 80 mg/ml but may differ by several mg/ml, thus including for example 92 mg/ml, 84 mg/ml, 88 mg/ml, 76 mg/ml, 72 mg/ml or 68 mg/ml. The skilled person is aware that such values are relative values that do not require a complete accuracy as long as the values approximately correspond to the recited values. Accordingly, a deviation from the recited value of for example 15%, more preferably of 10%, and most preferably of 5% is encompassed by the term “about”. These deviations of 15%, more preferably of 10% and most preferably of 5% hold true for all embodiments pertaining to this invention wherein the term “about” is used.

Preferably, the amount of sugar is exactly 80 mg/ml.

In accordance with the present invention, the solution according to step (b) of the method of the invention further comprises at least three different excipients selected from hydrophilic and amphiphilic excipients, wherein the excipients are characterized by polar, aliphatic, aromatic, negatively charged, and/or positively charged functional groups.

Excipients are well known in the art. Excipients are defined as ingredients that are included in a composition, such as e.g. pharmaceutical compositions, together with the active agent. They are typically added to formulations for several reasons and, thus, some excipients may have more than one effect or purpose for being part of the formulation. One of their main functions is that of a stabilizer. The main function of such stabilizers in pharmaceutical formulations is to protect the biologically active agent against the different types of stresses that are applied to said biologically active agent, such as e.g. a protein or a viral vector, during isolation, purification, drying e.g. by lyophilization, spray-drying, spray-freeze drying or foam-drying, storage either in solution or after drying as well as reconstitution after drying. There are specific mechanisms of stabilization of biologically active agents, which are specifically related to the excipients in the formulation. Stabilization is for example achieved by strengthening of the stabilizing forces, by destabilization of the denatured state, or by direct binding of excipients to the biologically active agents. Frequently employed excipients for use as stabilizers of biologically active agents include, without being limiting, sugars, polyols, amino acids, amines, salts, polymers and surfactants, each of which may exert different stabilizing effects.

Non-limiting examples of excipients selected from hydrophilic and amphiphilic excipients, wherein said excipients are further characterized by having polar, aliphatic, aromatic, negatively charged, and/or positively charged functional groups, classified according to international pharmacopoeias as save excipients for use in viral vector based compositions. Such excipients may for example be ALPHA-TOCOPHEROL, DL-1,2-DIMYRISTOYL-SN-GLYCERO-3-(PHOSPHO-S-(1-GLYCEROL)), 1,2-DIMYRISTOYL-SN-GLYCERO-3-PHOSPHOCHOLINE, 1,2-DISTEAROYL-SN-GLYCERO-3-(PHOSPHO-RAC-(1-GLYCEROL)), 1,2-DISTEAROYL-SN-GLYCERO-3-PHOSPHOCHOLINE, ACETIC ACID, ACETIC ACID, GLACIAL, ACETIC ANHYDRIDE, ACETONE SODIUM BISULFITE, ACETYLATED MONOGLYCERIDES, ACETYLTRYPTOPHAN, DL-ACTIVATED CHARCOAL, ADIPIC ACID, ALANINE, ALBUMIN AGGREGATED, ALBUMIN COLLOIDAL, ALBUMIN HUMAN, ALCOHOL, ALCOHOL, DEHYDRATED, DENATURED ALCOHOL, DILUTED ALCOHOL, AMMONIUM ACETATE, AMMONIUM HYDROXIDE, AMMONIUM SULFATE, ANHYDROUS CITRIC ACID, ANHYDROUS DEXTROSE, ANHYDROUS LACTOSE, ANHYDROUS TRISODIUM CITRATE, ARGININE, ASCORBIC ACID, ASPARTIC ACID, BENZALKONIUM CHLORIDE, BENZENESULFONIC ACID, BENZETHONIUM CHLORIDE, BENZOIC ACID, BENZYL ALCOHOL, BENZYL BENZOATE, BENZYL CHLORIDE, BIBAPCITIDE, BORIC ACID, BROCRINAT, BUTYLATED HYDROXYANISOLE, BUTYLATED HYDROXYTOLUENE, BUTYLPARABEN, CALDIAMIDE SODIUM, CALOXETATE TRISODIUM, CAPTISOL, CARBON DIOXIDE, CARBOXYMETHYLCELLULOSE, CARBOXYMETHYLCELLULOSE SODIUM, UNSPECIFIED FORM, CASTOR OIL, MICROCRISTALLINE CELLULOSE, CHLOROBUTANOL, CHLOROBUTANOL HEMIHYDRATE, ANHYDROUS CHLOROBUTANOL, CHOLESTEROL, CITRATE, CITRIC ACID, CITRIC ACID MONOHYDRATE, ANHYDROUS CITRIC ACID, CORN OIL, COTTONSEED OIL, CREATINE, CREATININE, CRESOL, CROSCARMELLOSE SODIUM, CROSPOVIDONE, CYSTEINE, CYSTEINE HYDROCHLORIDE, DALFAMPRIDINE, DEOXYCHOLIC ACID, DEXTRAN, DEXTRAN 40, DEXTROSE, DEXTROSE MONOHYDRATE, DEXTROSE SOLUTION, DIATRIZOIC ACID, DIETHANOLAMINE, DIMETHICONE MEDICAL FLUID 360, DIMETHYL SULFOXIDE, DIPALMITOYLPHOSPHATIDYLGLYCEROL, DL-DISODIUM HYDROGEN CITRATE, DISODIUM SULFOSALICYLATE, DISOFENIN, DISTEAROYLPHOSPHATIDYLCHOLINE, DL-DOCUSATE SODIUM, EDETATE DISODIUM, EDETATE DISODIUM ANHYDROUS, EDETATE SODIUM, EGG PHOSPHOLIPIDS, ETHANOLAMINE HYDROCHLORIDE, ETHYL ACETATE, ETHYLENEDIAMINE, ETHYLENE-VINYL ACETATE COPOLYMERS, EXAMETAZIME, FERRIC CHLORIDE, FRUCTOSE, GADOLINIUM OXIDE, GAMMA CYCLODEXTRIN, GELATIN, GENTISIC ACID, GENTISIC ACID ETHANOLAMIDE, GENTISIC ACID ETHANOLAMINE, GLUCEPTATE SODIUM, GLUCEPTATE SODIUM DIHYDRATE, GLUCONOLACTONE, GLUCURONIC ACID, GLUTATHIONE, GLYCERIN, GLYCINE, GLYCINE HYDROCHLORIDE, GUANIDINE HYDROCHLORIDE, HETASTARCH, HEXYLRESORCINOL, HISTIDINE, HUMAN ALBUMIN MICROSPHERES, HYALURONATE SODIUM, HYDROCHLORIC ACID, DILUTED HYDROCHLORIC ACID, HYDROXYETHYLPIPERAZINE ETHANE SULFONIC ACID, HYDROXYPROPYL .BETA.-CYCLODEXTRIN, IODINE, IODOXAMIC ACID, IOFETAMINE HYDROCHLORIDE, ISOLEUCINE, ISOPROPYL ALCOHOL, ISOTONIC SODIUM CHLORIDE SOLUTION, LACTIC ACID, DL-LACTIC ACID, L-LACTIC ACID, LACTOBIONIC ACID, LACTOSE MONOHYDRATE, LACTOSE, HYDROUS, LACTOSE, UNSPECIFIED FORM, LECITHIN, EGG LECITHIN, HYDROGENATED SOY LECITHIN, LEUCINE, LIDOFENIN, LYSINE, LYSINE ACETATE, MALEIC ACID, MANNITOL, MEBROFENIN, MEDRONATE DISODIUM, MEDRONIC ACID, MEGLUMINE, METACRESOL, METAPHOSPHORIC ACID, METHANESULFONIC ACID, METHIONINE, METHYL PYRROLIDONE, METHYLBORONIC ACID, METHYLCELLULOSES, METHYLENE BLUE, METHYLPARABEN, MIRIPIRIUM CHLORIDE, MONOTHIOGLYCEROL, N-(CARBAMOYL-METHOXY PEG-40)-1,2-DISTEAROYL-CEPHALIN SODIUM, N,N-DIMETHYLACETAMIDE, NIACINAMIDE, NIOXIME, NITRIC ACID, NITROGEN, OCTANOIC ACID, OXIDRONATE DISODIUM, OXYQUINOLINE, PALMITIC ACID, PEANUT OIL, PEG VEGETABLE OIL, PEG-20 SORBITAN ISOSTEARATE, PEG-40 CASTOR OIL, PEG-60 CASTOR OIL, PEG-60 HYDROGENATED CASTOR OIL, PENTASODIUM PENTETATE, PENTETIC ACID, PERFLUTREN, PHENOL, PHENOL, LIQUEFIED, PHENYLALANINE, PHENYLETHYL ALCOHOL, PHENYLMERCURIC NITRATE, EGG PHOSPHATIDYL GLYCEROL, EGG PHOSPHOLIPID, PHOSPHORIC ACID, POLOXAMER 188, POLYETHYLENE GLYCOL 200, POLYETHYLENE GLYCOL 300, POLYETHYLENE GLYCOL 3350, POLYETHYLENE GLYCOL 400, POLYETHYLENE GLYCOL 4000, POLYETHYLENE GLYCOL 600, POLYGLACTIN, POLYLACTIDE, POLYOXYETHYLENE FATTY ACID ESTERS, POLYOXYL 35 CASTOR OIL, POLYPROPYLENE GLYCOL, POLYSILOXANE, POLYSORBATE 20, POLYSORBATE 40, POLYSORBATE 80, POLYVINYL ALCOHOL, POTASSIUM BISULFITE, POTASSIUM CHLORIDE, POTASSIUM HYDROXIDE, POTASSIUM METABISULFITE, POTASSIUM PHOSPHATE, DIBASIC, POTASSIUM PHOSPHATE, MONOBASIC, POVIDONE K12, POVIDONE K17, POVIDONES, PROLINE, PROPYL GALLATE, PROPYLENE GLYCOL, PROPYLPARABEN, PROTAMINE SULFATE, SACCHARIN SODIUM, SACCHARIN SODIUM ANHYDOUS SALT, SERINE, SESAME OIL, SILICONE, SIMETHICONE, SODIUM ACETATE, SODIUM ACETATE ANHYDROUS, SODIUM ASCORBATE, SODIUM BENZOATE, SODIUM BICARBONATE, SODIUM BISULFATE, SODIUM BISULFITE, SODIUM CARBONATE, SODIUM CARBONATE DECAHYDRATE, SODIUM CARBONATE MONOHYDRATE, SODIUM CHLORATE, SODIUM CHLORIDE, SODIUM CHLORIDE INJECTION, SODIUM CHLORIDE INJECTION, BACTERIOSTATIC, SODIUM CHOLESTERYL SULFATE, SODIUM CITRATE, SODIUM DESOXYCHOLATE, SODIUM DITHIONITE, SODIUM FORMALDEHYDE SULFOXYLATE, SODIUM GLUCONATE, SODIUM HYDROXIDE, SODIUM HYPOCHLORITE, SODIUM IODIDE, SODIUM LACTATE, SODIUM LACTATE, L-SODIUM METABISULFITE, SODIUM OLEATE, SODIUM PHOSPHATE, SODIUM PHOSPHATE DIHYDRATE, DIBASIC SODIUM PHOSPHATE, DIBASIC SODIUM PHOSPHATE, DIBASIC SODIUM PHOSPHATE, SODIUM PHOSPHATE, DIBASIC, DODECAHYDRATE, DIBASIC SODIUM PHOSPHATE, MONOBASIC SODIUM PHOSPHATE, MONOBASIC ANHYDROUS SODIUM PHOSPHATE, MONOBASCIC SODIUM PHOSPHATE, MONOBASIC SODIUM PHOSPHATE, SODIUM PHOSPHITE, SODIUM PYROPHOSPHATE, SODIUM SUCCINATE HEXAHYDRATE, SODIUM SULFATE, ANHYDROUS SODIUM SULFATE ANHYDROUS, SODIUM SULFITE, SODIUM TARTRATE, SODIUM THIOGLYCOLATE, SODIUM THIOMALATE, SODIUM THIOSULFATE, SODIUM THIOSULFATE ANHYDROUS, SODIUM TRIMETAPHOSPHATE, SORBITAN MONOPALMITATE, SORBITOL, SORBITOL SOLUTION, SOYBEAN OIL, STANNOUS CHLORIDE, ANHYROUS STANNOUS CHLORIDE, STANNOUS FLUORIDE, STANNOUS TARTRATE, STARCH, STEARIC ACID, STERILE WATER FOR INHALATION, STERILE WATER FOR INJECTION, SUCCIMER, SUCCINIC ACID, SUCROSE, SULFOBUTYLETHER, BETA-CYCLODEXTRIN, SULFUR DIOXIDE, SULFURIC ACID, SULFUROUS ACID, TARTARIC ACID, DL TARTARIC ACID, TERT-BUTYL ALCOHOL, TETRAKIS(2-METHOXYISOBUTYLISOCYANIDE)COPPER(I) TETRAFLUOROBORATE, TETROFOSMIN, THEOPHYLLINE, THIMEROSAL, THREONINE, TIN, TRIFLUOROACETIC ACID, TRISODIUM CITRATE DIHYDRATE, TROMANTADINE, TROMETHAMINE, TRYPTOPHAN, TYROSINE, UREA, URETHANE, VALINE, VERSETAMIDE, and/or YELLOW WAX.

In a highly preferred embodiment, the at least three different excipients comprise at least one amino acids or the at least three different excipients are three different amino acids.

Preferred amounts of the sum of excipients to be comprised in the solution according to the invention are between 0.001 and 100 mg/ml, preferably between 1 and 80 mg/ml, more preferably between 5 and 60 mg/ml, even more preferably between 10 and 30 mg/ml and most preferably the amount is about 20 mg/ml.

Preferably, the solution comprises trehalose or sucrose as the sugar and mannitol as the sugar alcohol and amino acids as the at least three excipients. Even more preferably, the solution comprises trehalose as the sugar and at least three different amino acids as the at least three excipients.

Furthermore, the solution is characterized by an excipient to sugar ratio of at least 1:2 (w/w). More preferably, the solution is characterized by an excipient to sugar ratio of at least 1:1.5 (w/w), such as e.g. at least 1:1 (w/w) and most preferably of at least 1:0.1 (w/w).

Preferably, the pH value of the resulting composition according to step (b) will be adjusted to pH values between 4.0 and 9.0 before mixing with the replication deficient viral vectors of step (a). The pH value chosen depends on the requirements for the particular viral vector, determined by biologic characteristics such as e.g. size, enveloped (lipid membrane) or not enveloped etc.

In a third step (c), the method of the present invention comprises the step of mixing the replication deficient viral vectors of step (a) with the solution of step (b).

The term “mixing”, as used herein, is not particularly limited and includes all means of mixing viral vectors with a solution according to (b). For example, the components of step (a) and (b) can simply be transferred into the same vessel, where they can mix by diffusion; they can additionally be stirred, e.g. by swirling the vessel around or by stirring with a suitable tool. Stirring can be for a limited amount of time, such as e.g. once or twice, or can be continuously. Preferably, the components of step (a) and (b) can mixed together by re-buffering of the composition of the recited step (a) in the composition of the recited step (b) using chromatographic operations as well as dialysis, ultrafiltration and diafiltration operations.

The order of steps (a) and (b) is not particularly limited, i.e. step (a) can be carried out first, followed by step (b), or vice versa. Moreover, steps (a) and (b) can be carried out concomitantly. Step (c) is then carried out after steps (a) and (b) have been carried out.

In one embodiment, the method of the present invention consists of the recited steps (a) to (c). However, it will be appreciated that where the method of the invention comprises (rather than consists of) the cited steps (a) to (c), further method steps may be included in the method. For example, additional washing and/or drying steps may be included. Preferably, the method of the invention consists of the cited steps (a) to (c), optionally in combination with the below described additional method steps (d) and (e), and optionally in combination with additional washing steps. Even more preferably, the method of the present invention consists of the recited steps (a) to (c), in combination with the below described additional method steps (d) drying and (e) reconstitution of the resulting dried composition.

In accordance with the present invention, a method is provided for the preparation of improved viral vector-based vaccines and gene transfer therapeutics. By preparing vector-based compositions using the method of the present invention, unappreciated polydispersity can be avoided, thus resulting in a more suitable ratio between vector particle distribution and functional efficacy. Moreover, as discussed herein above, low polydispersity is associated with lower viscosity and not only provides better infectivity, but also leads to better syringeability and injectability.

In a preferred embodiment of the method of the invention, the at least three different excipients comprise amino acids. In an even more preferred embodiment of the method of the invention, the at least three different excipients are at least three different amino acids.

The term “amino acid”, as used herein, is well known in the art. Amino acids are the essential building blocks of proteins. In accordance with the present invention, the term “amino acid” refers to free amino acids which are not bound to each other to form oligo- or polymers such as dipeptides, tripeptides, oligopeptides or proteins (also referred to herein as polypeptides). The term “amino acid” includes naturally occurring amino acids, but also other amino acids such as artificial amino acids. They can be classified into the characteristic groups of excipients with non-polar, aliphatic; polar, uncharged; positively and/or negatively charged and/or aromatic R groups (Nelson D. L. & Cox M. M., “Lehninger Biochemie” (2005), pp. 122-127). The amino acids comprised in the solution (b) of the present invention can be selected from naturally occurring amino acids as well as artificial amino acids or derivatives of these naturally occurring or artificial amino acids.

Naturally occurring amino acids include the 20 amino acids that make up proteins (i.e. the so-called proteinogenic amino acids), i.e. glycine, proline, arginine, alanine, asparagine, aspartic acid, glutamic acid, glutamine, cysteine, phenylalanine, lysine, leucine, isoleucine, histidine, methionine, serine, valine, tyrosine, threonine and tryptophan. Other naturally occurring amino acids are e. g. carnitine, creatine, creatinine, guanidinoacetic acid, ornithine, hydroxyproline, homocysteine, citrulline, hydroxylysine or beta-alanine. Artificial amino acids are amino acids that have a different side chain length and/or side chain structure and/or have the amine group at a site different from the alpha-C-atom. Derivates of amino acids include, without being limiting, n-acetyl-tryptophan, phosphonoserine, phosphonothreonine, phosphonotyrosine, melanin, argininosuccinic acid and salts thereof and DOPA. In connection with the present invention, all these terms also include the salts of the respective amino acids.

In a preferred embodiment the at least three amino acids provided in the solution according to the invention are not more than four amino acids or not more than three amino acids. Thus, the solution may comprise only three or only four amino acids.

In an embodiment of the invention, the at least three different amino acids are selected from at least two different groups of
(a) amino acids with non polar, aliphatic R groups;
(b) amino acids with polar, uncharged R groups;
(c) amino acids with positively charged R groups;
(d) amino acids with negatively charged R groups; and
(e) amino acids with aromatic R groups.

The at least three different amino acids may also be selected from at least three different groups (a) to (e). In a further embodiment, the solution comprises four different amino acids selected from four different groups (a) to (e).

In a preferred embodiment of the invention, the at least three amino acids, at least provide one anti-oxidative functional group and at least one osmolytic function and at least one buffering function and at least one charged functional group. The charged functional group may be a positively or negatively charged functional group

The term “amino acids that provide an osmolytic function”, as used herein, relates to amino acids with that provide an osmolytic property. Such amino acids are also well-known in the art and include, for example, glycine, alanine, and glutamic acid, as well as derivatives of proteinogenic and non-proteinogenic amino acids, respectively, such as for example, betaine, carnitine, creatine, creatinine, and R-alanine.

The term “amino acids that provide an anti-oxidative functional group”, as used herein, relates to amino acids that provide an anti-oxidative property via (one of) their side chain(s). Such amino acids are also well-known in the art and include, for example, methionine, cysteine, histidine, tryptophan, phenylalanine, and tyrosine, as well as derivatives of proteinogenic and non-proteinogenic amino acids such as for example N-acetyl-tryptophan, N-acetyl-histidine, or carnosine.

The term “amino acids that provide a buffering function” relates to amino acids that provide a buffering capacity via one or more of their functional groups. Such amino acids are also well-known in the art and include, for example, glycine, arginine, and histidine.

In a preferred embodiment of the invention, the amino acids comprised in the solution and/or composition are histidine, glutamine and methionine. In another preferred embodiment of the invention, the amino acids comprised in the solution and/or composition are histidine, lysine and methionine. In another preferred embodiment of the invention, the amino acids comprised in the solution and/or composition are histidine, alanine, glycine, glutamine, and methionine. In another preferred embodiment of the invention, the amino acids comprised in the solution and/or composition are histidine, alanine, and glutamine. Thus, in a preferred embodiment of the invention, the amino acids comprised in the solution and/or composition may not comprise methionine. In another preferred embodiment of the invention, the amino acids comprised in the solution and/or composition are histidine, glycine, and methionine. In another preferred embodiment of the invention, the amino acids comprised in the solution and/or composition are histidine, glycine, methionine, alanine, and lysine. In another preferred embodiment of the invention, the amino acids comprised in the solution and/or composition are histidine, glycine, methionine, and alanine.

In an embodiment of the method of the invention, the at least three different excipients comprise “at least one dipeptide and/or tripeptide”. Where more than one di- or tripeptide is comprised in the solution, a mixture of dipeptides and tripeptides is explicitly envisaged herein. The number of di- and tripeptides can be selected independently of each other, e.g. the solution may comprise two dipeptides and three tripeptides. It will be readily understood by the skilled person that when referring to a certain number of di- and tripeptides herein, said number is intended to limit the amount of different types of di- and tripeptides, but not the number of molecules of one type of dipeptide or tripeptide. Thus, for example the term “four dipeptides or tripeptides”, refers to four different types of dipeptides and/or tripeptides, wherein the amount of each individual di- and/or tripeptide is not particularly limited. Preferably, the number of (different) di- or tripeptides does not exceed nine di- or tripeptides.

The term “dipeptide or tripeptide”, as used herein, relates to peptides consisting of two or three amino acids, respectively. Exemplary dipeptides are glycylglutamine (Gly-Gln), glycyltyrosine (Gly-Tyr), alanylglutamine (Ala-Gln) and glycylglycine (Gly-Gly). Further non-limiting examples of naturally occurring dipeptides are carnosine (beta-alanyl-L-histidine), N-acetyl-carnosine (N-acetyl-(beta-alanyl-L-histidine), anserine (beta-alanyl-N-methyl histidine), homoanserine (N-(4-aminobutyryl)-L-histidine), kyotorphin (L-tyrosyl-L-arginine), balenine (or ophidine) (beta-alanyl-N tau-methyl histidine), glorin (N-propionyl-γ-L-glutamyl-L-ornithine-δ-lac ethyl ester) and barettin (cyclo-[(6-bromo-8-en-tryptophan)-arginine]). Examples of artificial dipeptides include, without being limiting, aspartame (N-L-a-aspartyl-L-phenylalanine 1-methyl ester) and pseudoproline.

Exemplary tripeptides are glutathione (γ-glutamyl-cysteinyl-glycine) and its analogues ophthalmic acid (L-γ-glutamyl-L-α-aminobutyryl-glycine) as well as norophthalmic acid (γ-glutamyl-alanyl-glycine). Further non-limiting examples of tripeptides include isoleucine-proline-proline (IPP), glypromate (Gly-Pro-Glu), thyrotropin-releasing hormone (TRH, thyroliberin or protirelin: L-pyroglutamyl-L-histidinyl-L-prolinamide), melanostatin (prolyl-leucyl-glycinamide), leupeptin (N-acetyl-L-leucyl-L-leucyl-L-argininal) and eisenin (pGlu-Gln-Ala-OH).

In an alternative embodiment, the at least three different excipients may not comprise a dipeptide and/or tripeptide.

It is also envisaged herein that the solution of (b) comprises at least three excipients including (an) amino acid(s) as well as at least one di- and/or tripeptide.

Preferably, the total amount of all amino acids, dipeptides and/or tripeptides (that is the sum of all of these components in the solution) to be employed is between 0.001 and 100 mg/ml, preferably between 1 and 80 mg/ml, more preferably between 5 and 60 mg/ml, even more preferably between 10 and 30 mg/ml and most preferably the amount is about 20 mg/ml.

In an embodiment of the invention, the concentration of single amino acids may be between 0.1 mg/ml to 60 mg/ml, preferably the concentration of single amino acids may be between 0.3 mg/ml to 50 mg/ml, more preferably between 0.5 mg/ml to 40 mg/ml. For example, the concentration of alanine may preferably be between 5 mg/ml to 30 mg/ml, more preferably between 7.5 mg/ml to 25 mg/ml. For example, the concentration of glycine may preferably be between 5 mg/ml to 30 mg/ml, more preferably between 7.5 mg/ml to 25 mg/ml. For example, the concentration of lysine hydrochloride may preferably be between 10 mg/ml to 40 mg/ml, more preferably between 20 mg/ml to 35 mg/ml. For example, the concentration of histidine may preferably be between 0.5 mg/ml to 15 mg/ml, more preferably between 1 mg/ml to 10 mg/ml, most preferably between 2 mg/ml to 5 mg/ml. For example, the concentration of glutamine may preferably be between 0.5 mg/ml to 20 mg/ml, more preferably between 1 mg/ml to 15 mg/ml, most preferably between 1.5 mg/ml to 12.5 mg/ml. For example, the concentration of methionine may preferably be between 0.1 mg/ml to 10 mg/ml, more preferably between 0.25 mg/ml to 7.5 mg/ml, most preferably between 0.5 mg/ml to 5 mg/ml.

It is preferred that the amino acids, and/or the di- and/or tripeptides, when used in connection with medical applications, do not exert any pharmacological properties.

In a preferred embodiment, the solution and/or composition provided in step b) may not comprise a surfactant. In another preferred embodiment, the solution or composition may not comprise polyvinyl pyrrolidone and/or polyoxyethylene-polyoxypropylene block copolymer (Pluronic® F-68).

It has been shown in Examples 1 and 2 that solutions as described above comprising a viral vector may be stored in liquid state over extended periods of time with only a limited loss of transfection activity.

Accordingly, in another preferred embodiment of the method of the invention, the method comprises a further step (d) of storing the composition obtained in step (c) by mixing the replication deficient viral vectors of step (a) with the solution of step (b) in liquid state.

Preferably the composition obtained in step (c) is stored in liquid state for at least 30 days, more preferably at least 6 months, even more preferably 9 months and most preferably at least 12 months. For example, the composition may be stored in liquid state between 9 and 18 months. The composition may also be stored for 24 months.

The composition obtained in step (c) may preferably be stored in a liquid state for the periods indicated above at temperature between 4° and 30°, preferably between 10° C. and 28° C., most preferably between 20° C. and 27° C. In one embodiment, the composition is stored at about 25° C.

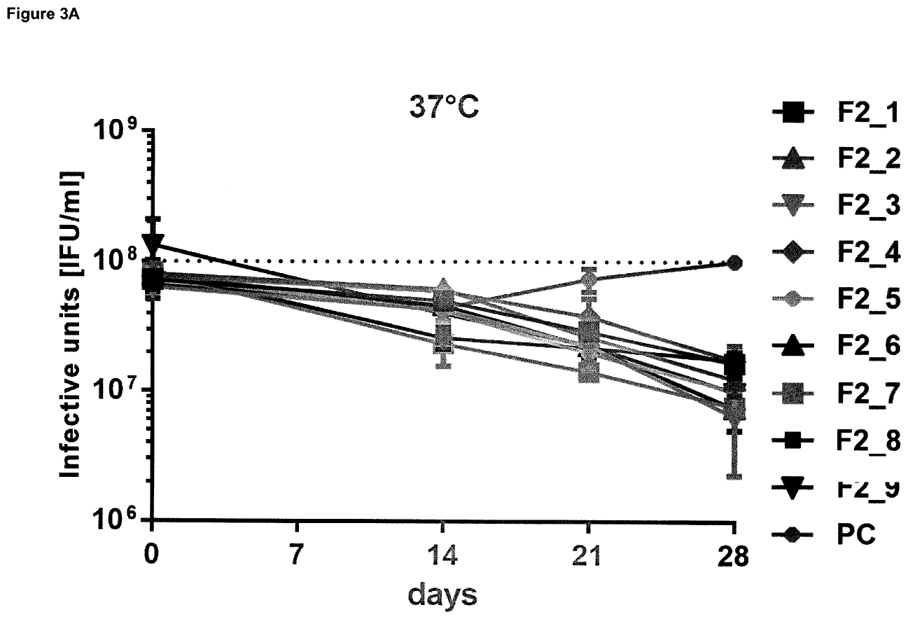

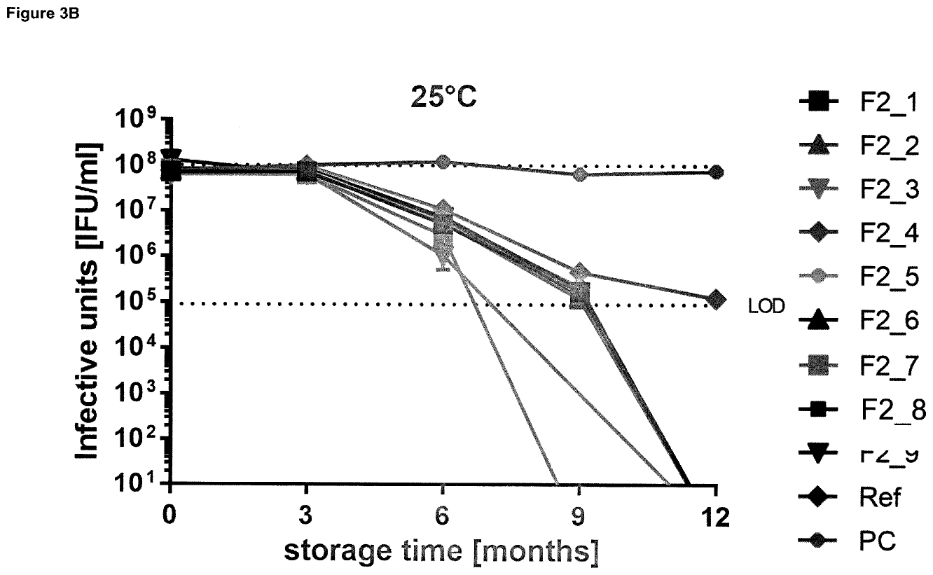

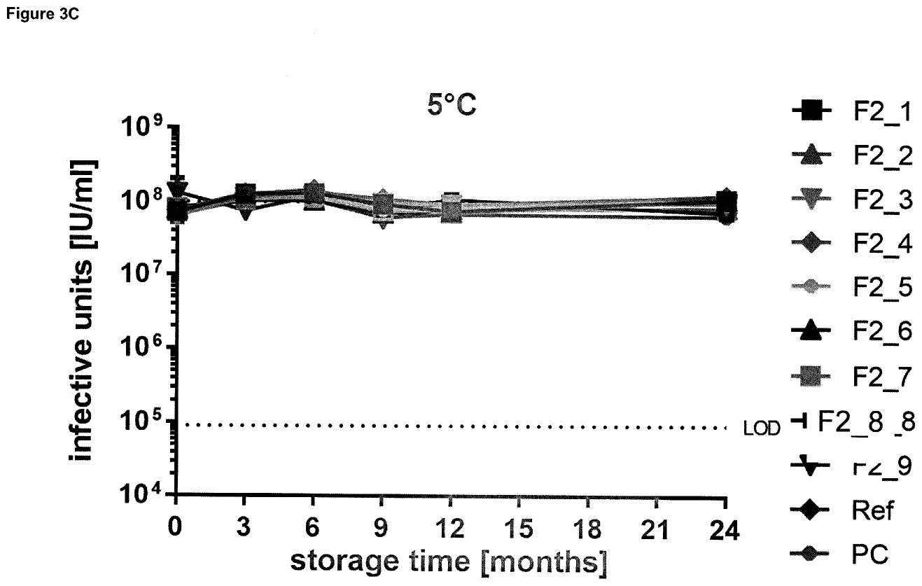

As shown in Example 2 and the related FIG. 3, storage of the composition in step (d) of the method of the invention for 6 months at 25° C. may lead to a loss of infectivity titer of no more than between 1.5 and 2 log levels. Most surprisingly, during storage of the composition over 12 months at 25° C. in step (d) of the method of the invention, the loss in infectivity titer of the viral vector comprised in the composition may be no more than 3 log levels.

In another preferred embodiment of the method of the invention, the viral vector-based composition is prepared for storage as a dried preparation. Such composition comprising viral vectors (in liquid or dried preparations) may be subsequently used for the preparation of vaccines or gene transfer therapeutics.

In a further preferred embodiment of the method of the invention, which embodiment comprises a further step of drying the composition obtained in step (c), the composition is dried by freeze-drying, spray-drying, spray freeze drying, or supercritical drying.

The term “drying”, as used herein, refers to the reduction or removal of the liquid content present in the composition. The liquid content is considered to have been reduced if the liquid is reduced to less than 20%, such as for example less than 10%, such as for example less than 8%, more preferably less than 7%, such as less than 5% or less than 1%. Even more preferably, the liquid is reduced to 0.5% or less.

Suitable methods for drying include, without being limiting, lyophilisation (freeze drying), spray drying, freeze-spray drying, convection drying, conduction drying, gas stream drying, drum drying, vacuum drying, dielectric drying (by e.g. radiofrequency or microwaves), surface drying, air drying or foam drying.

Freeze-drying, also referred to as lyophilisation, is also well known in the art and includes the steps of freezing the sample and subsequently reducing the surrounding pressure while adding sufficient heat to allow the frozen water in the material to sublime directly from the solid phase to the gas phase followed by a secondary drying phase. Preferably, the lyophilised preparation is then sealed to prevent the re-absorption of moisture.

Spray-drying is also well known in the art and is a method to convert a solution, suspension or emulsion into a solid powder in one single process step. Generally, a concentrate of the liquid product is pumped to an atomising device, where it is broken into small droplets. These droplets are exposed to a stream of hot air and lose their moisture very rapidly while still suspended in the drying air. The dry powder is separated from the moist air in cyclones by centrifugal action, i.e. the dense powder particles are forced toward the cyclone walls while the lighter, moist air is directed away through the exhaust pipes.

Spray-drying is often the method of choice, as it avoids the freezing step and requires lower energy costs as compared to lyophilisation. Spray-drying has also been shown to be a particularly advantageous drying procedure that is suitable for biomolecules, due to the short contact time with high temperature and its special process control. Thus, because spray-drying results in a dispersible dry powder in just one step it is often favoured to freeze drying when it comes to drying techniques for biomolecules.

Spray-freeze-drying is also well known in the art and is a method that combines processing steps common to freeze-drying and spray-drying. The sample provided is nebulised into a cryogenic medium (such as e.g. liquid nitrogen), which generates a dispersion of shock-frozen droplets. This dispersion is then dried in a freeze dryer.

Supercritical drying is another technique well known in the art. This method relies on high-temperature and high-pressure above the critical temperature (T_{c}) and critical pressure (p_{c}) to change a liquid into a gas wherein no phase boundaries are crossed but the liquid to gas transition instead passes through the supercritical region, where the distinction between gas and liquid ceases to apply. The densities of the liquid phase and vapour phase become equal at the critical point of drying.

The step of drying the composition obtained in (c) may be by lyophilisation.

The method may further comprise the step of subsequently storing the composition comprising viral vectors at a temperature selected from about −90° C. to about 50° C. More preferably, the composition comprising viral vectors is subsequently stored at a temperature range selected from the group consisting of about −90° C. to about −70° C., about −30° C. to about −10° C., about 1° C. to about 10° C., about 15° C. to about 25° C. and about 30° C. to about 50° C. Even more preferably, the composition comprising viral vectors is subsequently stored at a temperature range selected from the group consisting of about −85° C. to about −75° C., about −25° C., to about −15° C., about 2° C. to about 8° C. and about 20° C. to about 45° C. Most preferably, the composition comprising viral vectors is subsequently stored at a temperature selected from about −80° C., about −20° C., room temperature, about 4° C., about 25° C. and about 40° C.

In a further preferred embodiment of the method of the invention, the method further comprises a step (e) of reconstituting the composition obtained after drying.

Reconstituting of the composition can be carried out by any means known in the art, such as e.g. dissolving the dried composition in a suitable solution. Non-limiting examples of suitable solutions include the solution of step (b) used for mixing with the viral vector as well as any other solution known to be suitable for compositions comprising viral vectors, such as e.g. water for injection, buffered solutions, solutions comprising amino acids, sugars, buffers, surfactants or mixtures thereof.

According to the present invention, the viral vector is a viral vector for vaccination or gene therapy. The vaccination may be a prophylactic or a therapeutic vaccination. A “gene therapy” may for example be a therapy wherein a mutated gene that causes disease is replaced with a healthy copy of the gene, a therapy wherein a mutated gene that is functioning improperly is inactivated or knocked out, or a new gene is introduced in to a cell cure a disease. According to the present invention the gene therapy may be a cell therapy wherein a viable cell into which a new gene has been introduced, a mutated gene has been replaced, inactivated or knocked by means of a viral vector are injected, grafted or implanted into a patient in order to effectuate a medicinal effect. Preferably, the viable cell is a T cell type of leukocyte.

In a further preferred embodiment of the method of the invention, the viral vector is selected from the group consisting of adenovirus, Adenovirus-associated virus (AAV), lentivirus, vesicular stomatitis virus (VSV), MVA, or herpesviruses.

The Modified Vaccinia Ankara (MVA) virus is a highly attenuated strain of vaccinia virus that was developed towards the end of the campaign for the eradication of smallpox in the seventies of the previous century. MVA was derived from Vaccinia strain Ankara by over 570 passages in chicken embryo fibroblast cells (CEF). This resulted in six major deletions corresponding to the loss of about 10% of the vaccinia genome. The complete genomic sequence is known and has a length of 178 kp corresponding to 177 genes. The numerous mutations explain the attenuated phenotype of MVA and its inability to replicate in mammalian cells. MVA is widely considered as the Vaccinia virus strain of choice for clinical investigation because of its high safety profile. MVA has been administered to numerous animal species including monkeys, mice, swine, sheep, cattle, horses, and elephants, with no local or systemic adverse effects. Over 120,000 humans have been safely and successfully vaccinated against smallpox with MVA by intradermal, subcutaneous, or intramuscular injections. Studies in mice and nonhuman primates have further demonstrated the safety of MVA under conditions of immune suppression. Compared to replicating vaccinia viruses, MVA provides similar or higher levels of recombinant gene expression even in non-permissive cells. In animal models, recombinant MVA vaccines have been found immunogenic and to protect against various infectious agents including influenza, parainfluenza, measles virus, flaviviruses, and plasmodium parasites. The combination of a very good safety profile and the ability to deliver antigens in a highly immunogenic way makes MVA suitable as a vaccine vector.

Adenoviruses are medium-sized (90-100 nm), nonenveloped (naked) icosahedral viruses composed of a nucleocapsid and a double-stranded linear DNA genome. There are over 51 different serotypes in humans, which are responsible for 5-10% of upper respiratory infections in children, and many infections in adults as well. When these viruses infect a host cell, they introduce their DNA molecule into the host. The genetic material of the adenoviruses is not incorporated (transient) into the host cell's genetic material. The DNA molecule is left free in the nucleus of the host cell, and the instructions in this extra DNA molecule are transcribed just like any other gene. The only difference is that these extra genes are not replicated when the cell is about to undergo cell division so the descendants of that cell will not have the extra gene. As a result, treatment with the adenovirus will require re-administration in a growing cell population although the absence of integration into the host cell's genome should prevent the type of cancer seen in the SCID trials. This vector system has shown real promise in treating cancer and indeed the first gene therapy product (Gendicine) to be licensed is an adenovirus to treat cancer.

Viruses of the family adenoviridae infect various species of animals, including humans. Adenoviruses represent the largest non-enveloped viruses because they are the maximum size able to be transported through the endosome (i.e. envelope fusion is not necessary). The virion also has a unique “spike” or fiber associated with each penton base of the capsid that aids in attachment to the host cell via the coxsackie-adenovirus receptor on the surface of the host cell.

Adeno-associated virus (AAV) is a small virus that infects humans and some other primate species. AAV is not currently known to cause disease and, consequently, the virus causes a very mild immune response. AAV can infect both dividing and non-dividing cells and can incorporate its genome into that of the host cell. Moreover, episomal AAV elicits long and stable expression and, thus, AAV is suitable for creating viral vectors for gene therapy. Because of its potential use as a gene therapy vector, AAV has previously been modified (self-complementary adeno-associated virus; scAAV). Whereas AAV packages a single strand of DNA and requires the process of second-strand synthesis, scAAV packages both strands which anneal together to form double stranded DNA. This approach allows for rapid expression in the target cell.

Lentiviruses, a subclass of retroviruses have recently been adapted as viral vectors for gene delivery because of their unique ability to integrate into the genome of non-dividing cells. The viral genome in the form of RNA is reverse-transcribed when the virus enters the cell to produce DNA, which is then inserted into the genome by the viral integrase enzyme. The vector, now called a provirus, remains in the genome and is passed on to the progeny of the cell when it divides.

Vesicular stomatitis Indiana virus (VSIV) (often still referred to as VSV) is a virus in the family Rhabdoviridae; the well-known rabies virus belongs to the same family. VSIV can infect insects, cattle, horses and pigs. It has particular importance to farmers in certain regions of the world where it can infect cattle and lead to diseases similar to the foot and mouth disease virus.

Herpes viruses belong to the Herpesviridae, a large family of DNA viruses that cause diseases in animals and humans. Herpes simplex viruses (HSV) HSV-1 and HSV-2 (orolabial herpes and genital herpes), Varicella zoster virus (VZV; chicken-pox and shingles), Epstein-Barr virus (EBV; mononucleosis), and Cytomegalovirus (CMV) are widespread among humans. More than 90% of adults have been infected with at least one of these, and a latent form of the virus remains in most people. Herpes viruses are currently used as gene transfer vectors due to their high transgenic capacity of the virus particle allowing to carry long sequences of foreign DNA, the genetic complexity of the virus genome, allowing to generate many different types of attenuated vectors possessing oncolytic activity, and the ability of HSV vectors to invade and establish lifelong non-toxic latent infections in neurons from sensory ganglia from where transgenes can be strongly and long-term expressed. Three different classes of vectors can be derived from HSV: replication-competent attenuated vectors, replication-incompetent recombinant vectors and defective helper-dependent vectors known as amplicons. Replication-defective HSV vectors are made by the deletion of one or more immediate-early genes, e.g. ICP4, which is then provided in trans by a complementing cell line. Oncolytic HSV vectors are promising therapeutic agents for cancer. Such HSV based vectors have been tested in glioma, melanoma and ovarian cancer patients.

It is particularly preferred that the viral vector is an adenovirus or AAV.

The above listed preferred viral vectors have been evaluated regarding their safety profile in animals and/or humans and preclinical and clinical data are available, respectively.

In another preferred embodiment of the method of the invention, the replication-deficient viral vector is a virus like particle.

Virus like particles (VLPs) provide the advantage that they are not infectious and do not contain viral genetic material. Accordingly, they are not associated with any risk of reassembly as is possible when live attenuated viruses are used as viral vectors.

In another preferred embodiment of the method of the invention, the method further comprises adding an antigenic polypeptide.

An “antigenic polypeptide” in accordance with the present invention is not particularly limited, as long as it elicits an immune response. The antigenic polypeptide can be selected from e.g. viruses, bacteria, or tumor cells. For example, the antigenic polypeptide can be a viral surface protein of a virus other than the viral vector employed in the method of the invention, or a part thereof; or a main immunogenic viral protein or part thereof. These additional antigenic polypeptides can for example be used for priming the immune system in a prime-boost vaccination. In that case, the boost reaction is elicited by the respective viral vector or VLP relied on for preparing the composition comprising viral vectors by the method of the present invention. The term “polypeptide” as used herein interchangeably with the term “protein” describes linear molecular chains of amino acids, including single chain proteins or their fragments.

The step of adding the antigenic polypeptide can be carried out at different time points. For example, the antigenic polypeptide can be added to the replication-deficient viral vector provided in step (a). Alternatively, the antigenic polypeptide can be additionally admixed in step (c) or be added to the resulting composition subsequently to the mixing in step (c). Furthermore, as an additional alternative, the antigenic polypeptide can be added to the composition comprising viral vectors after reconstitution in step (e).

In a further preferred embodiment of the method of the invention, the method further comprises adding at least one adjuvant.

Adjuvants as well as their mode of action are well known in the art. Some adjuvants, such as alum and emulsions (e.g. MF59e), function as delivery systems by generating depots that trap the antigenic substance at the injection site, providing slow release in order to provide a continued stimulation of the immune system. These adjuvants enhance the antigen persistence at the injection site and increase the recruitment and activation of antigen presenting cells (APCs). Particulate adjuvants (e.g. alum) have the capability to bind antigenic substances to form multi-molecular aggregates which will encourage uptake by APCs. Some adjuvants are also capable of directing antigen presentation by the major histocompatibility complexes (MHC). Other adjuvants, essentially ligands for pattern recognition receptors (PRR), act by inducing the innate immunity, predominantly targeting the APCs and consequently influencing the adaptive immune response. Members of nearly all of the PRR families are potential targets for adjuvants. These include Toll-like receptors (TLRs), NOD-like receptors (NLRs), RIG-I-like receptors (RLRs) and C-type lectin receptors (CLRs). They signal through pathways that involve distinct adaptor molecules leading to the activation of different transcription factors. These transcription factors (NF-κB, IRF3) induce the production of cytokines and chemokines and IL-18.

Preferably, the at least one adjuvant is selected from Alum, MF59®, AS03, AF03, AS04, RC-529, Virosomen, ISCOMATRIX®, CpG 1018, CpG 7909, VaxImmune, ProMune®, IC-31®, CTA1-DD or Cyclic di-AMP. These adjuvants, their class, indications and provider as well as product names are summarized in Table 2 below.

TABLE 1
Detailed informations on particularly preferred adjuvants.

| Adjuvant | Class | Main indications | Provider/Product |
|---|---|---|---|
| Alum | Aluminium salts | various | various |
| | Aluminiumhydroxid | | world-wide |
| | Aluminiumphosphate | | |
| | Aluminiumhydroxyphosphate | | |
| MF59 ® | Oil-in-Water emulsion | Seasonal | Novartis/Fluad |
| | 4.3% Squalen | Influenza | |
| | 0.5% Polysorbat 80 | | |
| | 0.5% Sorbitantriolate | | |
| | (Span 85 ®) | | |
| | 10 mM sodiumcitrate | | |
| AS03 | Oil-in-Water emulsion | Pandemic | GSK/Pandemrix |
| | 10.69 mg Squalene | Influenza | |
| | 11.86 mg D,L-α-Tocopherol | | |
| | (Vit. E) | | |
| | 4.86 mg Polysorbate 80 | | |
| AF03 | Oil-in-Water emulsion | Pandemic | Sanofi Pasteur/ |
| | 12.4 mg Squalene | Influenza | Humanza |
| | 1.9 mg Sorbitanoleate | | |
| | 2.4 mg Polysorbate 20 | | |
| | 2.3 mg Mannitol | | |
| AS04 | Kombination | Hepatitis B virus | GSK/Fendrix |
| | Monophposphoryllipid A und | Human | Cervarix |
| | Aluminiumsalz | Papillomavirus | |
| RC-529 | Combination synthetic | Hepatitis B virus | Dynavax |
| | monophposphoryllipid A and | | |
| | aluminiumsalt | | |
| Virosomen | Phosphatidylcholine bilayer | Hepatitis A virus | Crucell/ |
| | liposomes 150 nm | Seasonal | Inflexal V |
| | | Influenza | |
| ISCOMATRIX ® | ISCOM Immunostimulating | various | CSL Limited |
| | Complex | | Parkville, Victoria, |
| | Antigen | | Australien |
| | Cholesterol | | |
| | Phospholipid | | |
| | Saponin from Quillaja | | |
| | Saponaria | | |
| CpG 1018 | Oligodeoxynukleotide | Hepatitis B virus | Dynavax/ |
| | | Cancer | HEPLISAV-B |
| | | | SD-101 |
| CpG 7909 | Oligodeoxynukleotide | Cancer | Coley/Chiron/Pfizer |
| VaxImmune | | vaccination | GSK |
| ProMune ® | | Hepatitis B virus | |
| | | Treatment of | |
| | | Cancer | |
| IC-31 ® | Peptide and | Tuberkulosis | Intercell |
| | Oligodesoxynukleotid | | |
| CTA1-DD | Fusion protein from CTA1- | | MIVAC Development |
| | Domaine of Cholera Toxins | | AB in Sweden |
| | (CT) with maintaining | | |
| | ADP-ribosylating enzymatic | | |
| | function and a dimer from Ig | | |
| | binding domain of Protein A | | |
| | (S. aureus) as target domaine | | |

The step of adding the adjuvant can be carried out at different time points. For example, the adjuvant can be added to the replication-deficient viral vector provided in step (a). Alternatively, or additionally, the adjuvant can be admixed in step (c) or it can be added to the resulting composition subsequently to the mixing in step (c). As a further alternative or additional option, it can be added to the composition comprising viral vectors after reconstitution in step (e).

In another preferred embodiment of the method of the invention, at least one of the adjuvants is a saponine. Alternatively, the adjuvant is a mixture of substances comprising a saponine.

Saponines are a class of chemical compounds forming secondary metabolites which are found in natural sources, derived from natural sources or can be chemically synthesised. Saponines are found in particular abundance in various plant species. Saponines are amphipathic glycosides grouped phenomenologically by the soap-like foaming they produce when shaken in aqueous solutions, and structurally by their composition of one or more hydrophilic glycoside moieties combined with a lipophilic steroidal or triterpenoid aglycone. Their structural diversity is reflected in their physicochemical and biological properties. Non-limiting examples of saponines are glycyrrhizic acid, glycyrrhetinic acid, glucuronic acid, escin, hederacoside and digitonin.

Preferably, the saponine is selected from well-known adjuvant compositions, e.g., the saponine extracted from Quillaja saponaria, as listed in Table 1, without being limiting.

In another embodiment, the saponine is glycyrrhizic acid or a derivative thereof. Glycyrrhizic acid is also known as glycyrrhicic acid, glycyrrhizin or glycyrrhizinic acid. Glycyrrhizic acid is water-soluble and exists as an anion that can be a potential ligand to form electrostatically associated complexes with cationic molecules of active ingredients. Without wishing to be bound by theory, the present inventors hypothesise that the anionic glycyrrhizic acid forms complexes with amino acids present in the solution of the present invention (i.e. arginine, or lysine) through electrostatic interactions, hydrogen bonds or both. Derivatives of glycyrrhizic acid are well-known in the art and include those produced by transformation of glycyrrhizic acid on carboxyl and hydroxyl groups, by conjugation of amino acid residues into the carbohydrate part or the introduction of 2-acetamido-β-d-glucopyranosylamine into the glycoside chain of glycyrrhizic acid. Other derivatives are amides of glycyrrhizic acid, conjugates of glycyrrhizic acid with two amino acid residues and a free 30-COOH function and conjugates of at least one residue of amino acid alkyl esters in the carbohydrate part of the glycyrrhizic acid molecule. Examples of specific derivatives can be found e. g. in Kondratenko et al. (Russian Journal of Bioorganic Chemistry, Vol 30(2), (2004), pp. 148-153). Preferred amounts of glycyrrhizic acid (or derivatives thereof) to be employed are between 0.01 and 15 mg/ml, preferably between 0.1 and 10 mg/ml, more preferably between 0.5 and 5 mg/ml, even more preferably between 1 and 3 mg/ml and most preferably the amount is 2 mg/ml.

As is known in the art, saponines, in particular glycyrrhizic acid, have been found to be advantageously present in function of an adjuvant, as they enhance the immunogenic effect of the viral vector based composition.

In another preferred embodiment of the method of the invention, the viral vectors of (a) are viral vectors that have been reconstituted immediately after harvesting from cell cultures and purification.

Means and methods for reconstituting viral vectors are well known in the art. For example, after amplification of a replication-deficient viral vector, such as e.g. MVA, in the appropriate cell culture model, crude stock preparations of MVA can be semi-purified from cell debris and recombinant proteins by ultracentrifugation through a sucrose cushion. After discarding the supernatant (cell debris and sucrose) the pelleted viral vector material can be mixed with a solution according to (b). Alternatively, to obtain more highly purified viruses, the semi-purified material can be centrifuged through a 25-40% sucrose gradient. The viral vector band appearing at the lower half of the tube is concentrated and the remaining sucrose is simultaneously removed by filling an ultracentrifuge tube with the solution according to (b), pelleting the viral vector material by ultracentrifugation and suspending the pellet in a solution according to (b).

The decrease in the amount of infectious particles present in a composition as compared to non-infectious particles due to an increasing polydispersity starts immediately after harvesting viral particles from cell culture. Thus, it is particularly preferred in accordance with the present invention that the viral vectors are mixed with the solution of (b) as early as possible after the initial harvesting of the viral vectors.

According to a further aspect, the invention relates to a composition obtained or obtainable by the method of the invention.

Accordingly, the invention also relates to a composition comprising a viral vector and a solution as described above for use in the method according to the invention.

In one embodiment, the compositions may be characterized by a loss of infectivity titer of no more than between 1.5 and 2 log levels upon storage for 6 months at 25° C. In a further a further embodiment, the compositions may be characterized by a loss of infectivity titer of no more than 3 log levels upon storage of the composition over 12 months at 25° C. The infectivity titer of a viral vector sample may for example be determined in an infectivity assay by infecting cells with serial dilutions of the respective virus and detection of infection by immunostaining the infected cells with virus specific antibodies, for example immunostaining of the adenoviral hexon protein, as shown in Example 1.1.2.

These compositions may be used for anti-bacterial, antiviral, anti-cancer, anti-allergy vaccination and/or for gene transfer therapy for the treatment of diseases with a genetic background.

In a preferred embodiment, the composition is a pharmaceutical composition.

In accordance with the present invention, the term “pharmaceutical composition” relates to a composition for administration to a patient, preferably a human patient.

The pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgment of the ordinary clinician or physician. The pharmaceutical composition may be for administration once or for a regular administration over a prolonged period of time. Generally, the administration of the pharmaceutical composition should be in the range of for example 1 μg/kg of body weight to 50 mg/kg of body weight for a single dose. However, a more preferred dosage might be in the range of 10 μg/kg to 20 mg/kg of body weight, even more preferably 100 μg/kg to 10 mg/kg of body weight and even more preferably 500 μg/kg to 5 mg/kg of body weight for a single dose.

The components of the pharmaceutical composition to be used for therapeutic administration must be sterile. Sterility is readily accomplished for example by filtration through sterile filtration membranes (e.g., 0.2 μm membranes).

The various components of the composition may be packaged as a kit with instructions for use.

Accordingly, the present invention further relates to a kit comprising a composition comprising viral vectors obtained or obtainable by the method of the invention and, optionally, instructions how to use the kit.

Whereas the term “kit” in its broadest sense does not require the presence of any other compounds, vials, containers and the like other than the recited components, the term “comprising”, in the context of the kit of the invention, denotes that further components can be present in the kit. Non-limiting examples of such further components include antigenic polypeptides or adjuvants, as defined above, as well as preservatives, buffers for storage, enzymes etc.

Where several components are comprised in the kit, the various components of the kit may be packaged in one or more containers such as one or more vials. Consequently, the various components of the kit may be present in isolation or combination. The containers or vials may, in addition to the components, comprise preservatives or buffers for storage. In addition, the kit can contain instructions for use.

In a preferred embodiment of the kit of the invention, the kit comprises a composition comprising viral vectors obtained or obtainable by the method of the invention and, in the same or a separate container, an antigenic polypeptide. These separate containers with i) the composition comprising viral vectors and ii) the antigenic polypeptide can be used in separate vaccination steps (either simultaneously or subsequently to each other), e.g. for a prime-boost immunization approach.

In an alternative or additional preferred embodiment of the kit of the invention, the kit comprises a composition comprising viral vectors obtained or obtainable by the method of the invention and, in the same or a separate container, one or more adjuvants.

Also envisaged is a kit, comprising (i) a composition comprising viral vectors obtained or obtainable by the method of the invention; (ii) an antigenic polypeptide and (iii) one or more adjuvants, in the same or different containers.

The present invention also relates to the composition comprising viral vectors of the invention for use as a prime-boost vaccine.

The “prime-boost vaccine strategy” is well known in the art and encompasses a first step of “priming” an immune response, followed by a second step of “boosting” the previously primed immune response. This approach enables high levels of antigen specific T-cell memory as well as protective cellular immunity to pathogens, even in humans, and thus is a promising approach in vaccination (Woodland D L, Trends in Immunology, 2004; Nolz J C, Harty J T. Adv Exp Med Biol. 2011; 780:69-83. doi: 10.1007/978-1-4419-5632-3_7. Strategies and implications for prime-boost vaccination to generate memory CD8 T cells).

Compositions comprising viral vectors are highly attractive for therapeutic prime-boost vaccine approaches. For example, prophylactic vaccination for the prevention of HBV infection is well established. In contrast, an effective therapy of chronic hepatitis due to HBV infection and its sequelae is currently not available and might be successfully addressed by a prime-boost vaccination strategy with a specific antigen prime and a subsequent specific viral vector-based boost that induces antigen specific antibody production as well as antigen specific T cell responses both resulting in a highly efficient vaccination outcome. As discussed herein above, the data provided in the appended Examples show that the biological, immunogenic activity of a composition comprising viral vectors prepared by the method of the present invention is improved as compared to compositions comprising viral vectors prepared by other methods. In other words, the ability of the inventive compositions comprising viral vectors to stimulate the immune system of a subject, such as e.g. to elicit cytotoxic T lymphocytes (CTL) of the immune system to protect the subject against the disease for which the vaccine has been developed, is improved.

In a further preferred embodiment of the invention, the composition comprising viral vectors is for intramuscular, subcutaneous, intradermal, transdermal, oral, peroral, nasal, and/or inhalative application.

Unless otherwise defined, all technical and scientific terms used herein have the same meaning as commonly understood by one of ordinary skill in the art to which this invention belongs. In case of conflict, the patent specification, including definitions, will prevail.

Regarding the embodiments characterized in this specification, in particular in the claims, it is intended that each embodiment mentioned in a dependent claim is combined with each embodiment of each claim (independent or dependent) said dependent claim depends from.

For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a plurality of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The above considerations apply mutatis mutandis to all appended claims. To give a non-limiting example, the combination of claims 12, 8, 4, 3 and 1 is clearly and unambiguously envisaged in view of the claim structure. The same applies for example to the combination of claims 12, 8 and 1 etc.

The invention is illustrated with the following figures which show:

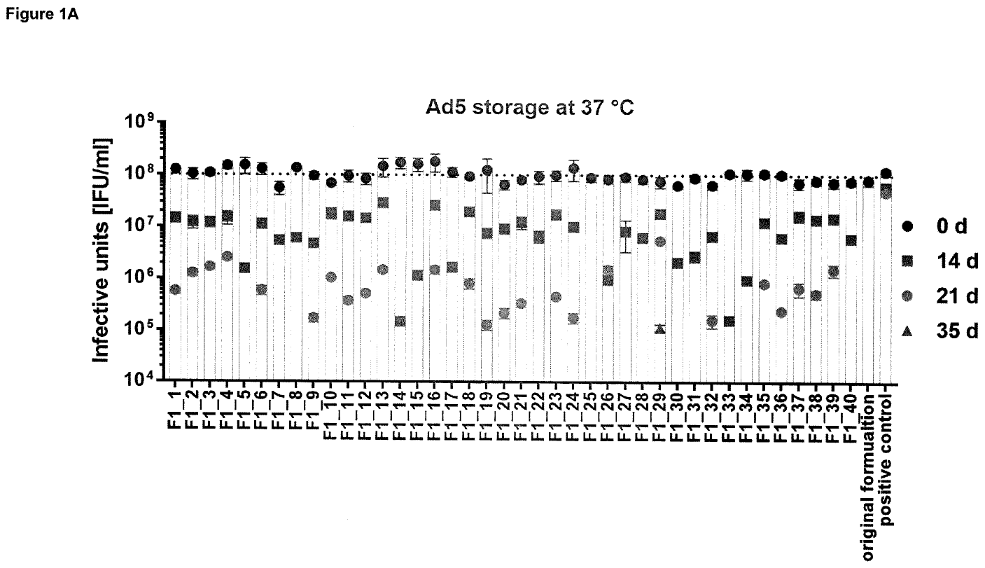

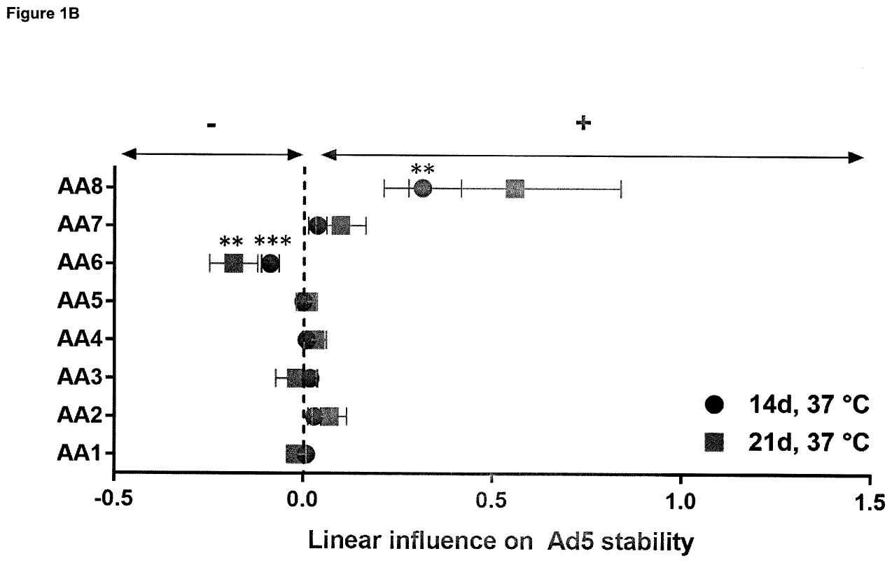

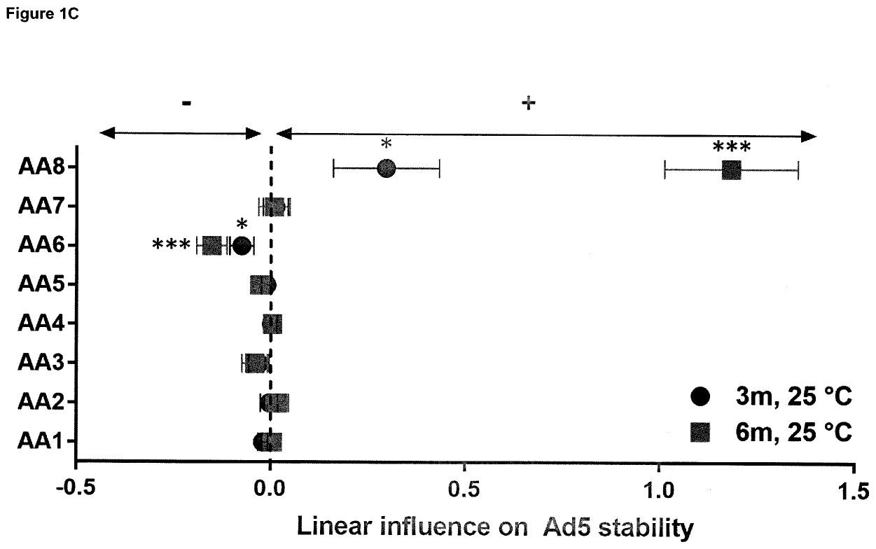

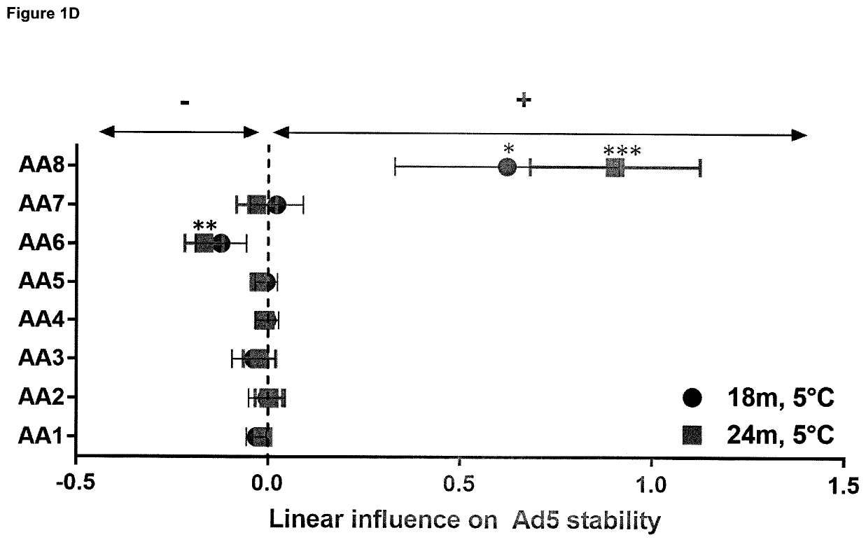

FIG. 1: (A) shows the infectivity of Ad5 in liquid formulations (round 1) after accelerated aging at 37° C.; (B) to (D) depict the linear influence on Ad5 stability (negative or positive) of single amino acids (AA) after short and long term storage.

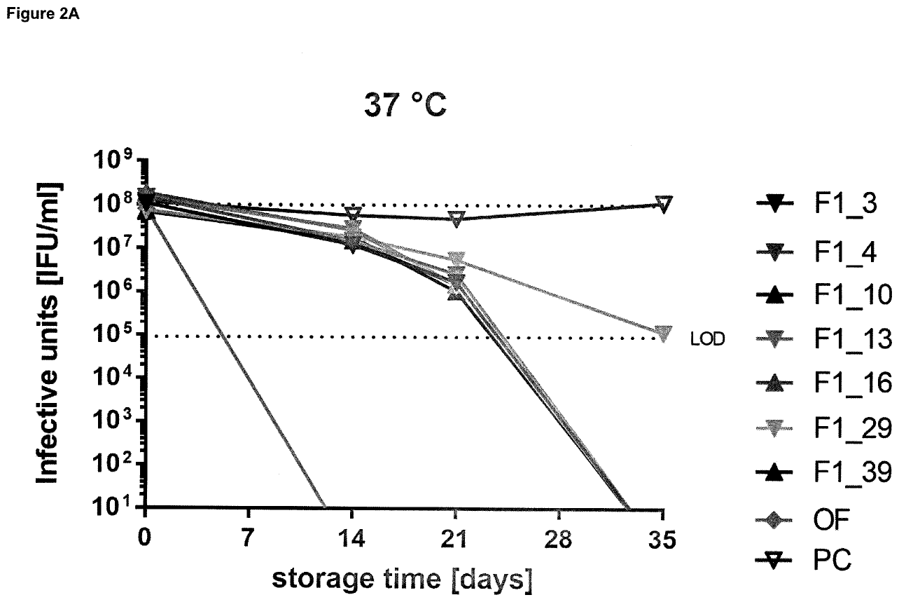

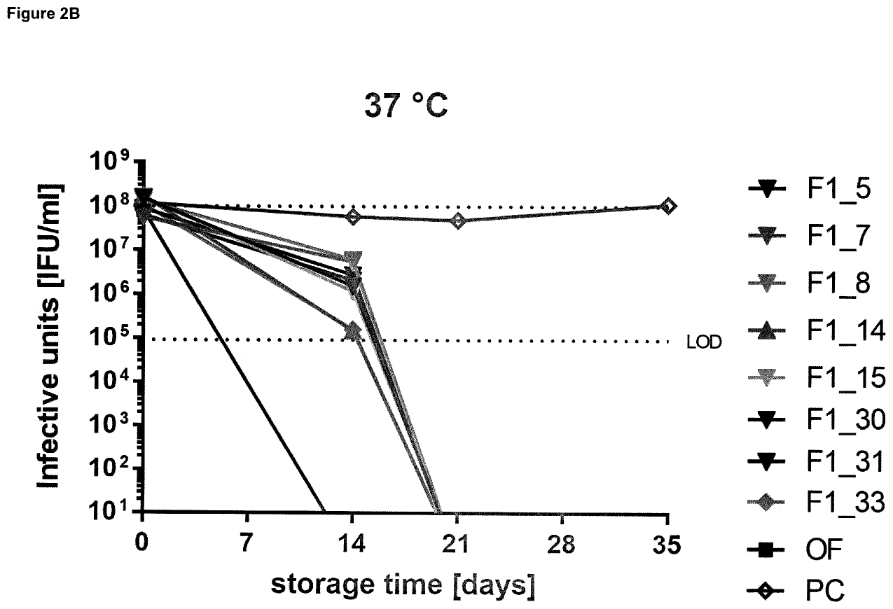

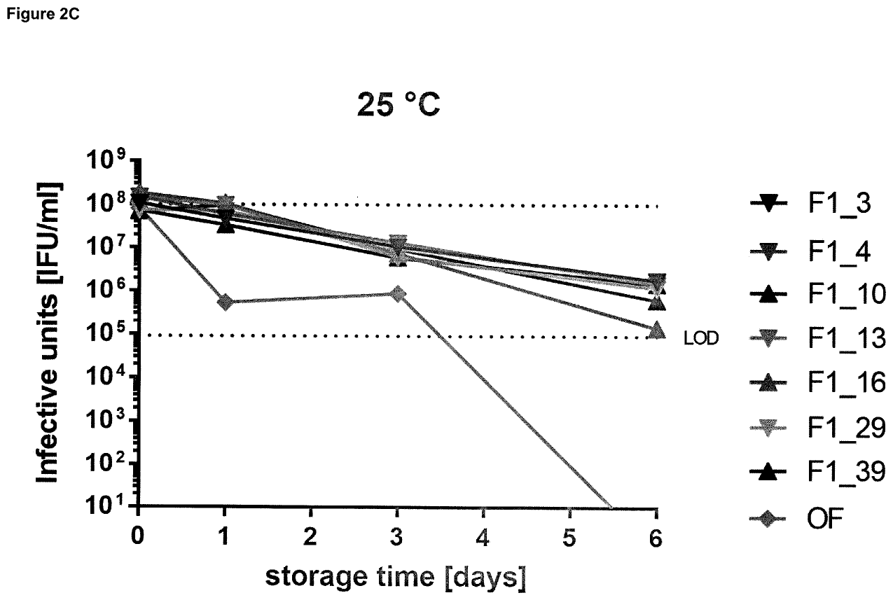

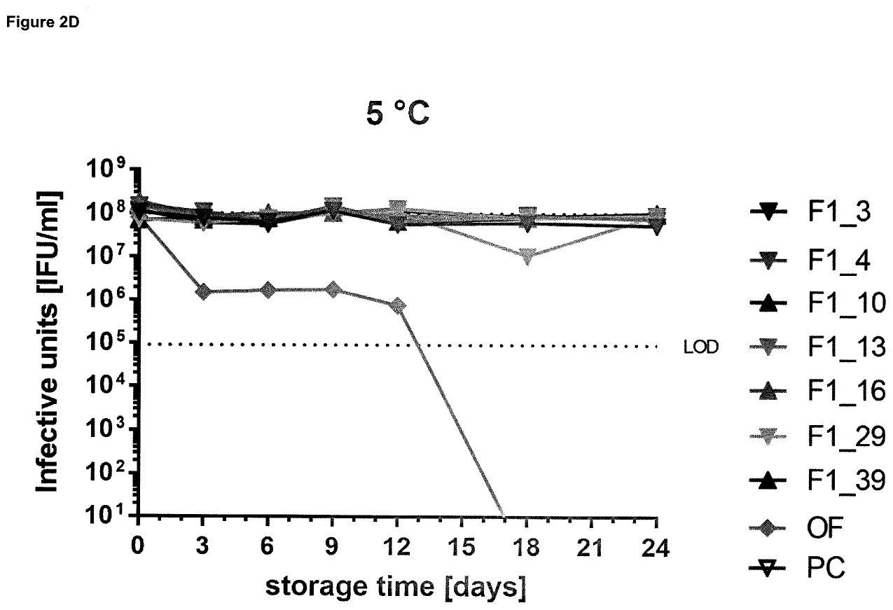

FIG. 2: (A) shows the infectivity of Ad5 in the best performing formulations after storage for up to 35 days at 37° C., (B) shows the infectivity of Ad5 in the worst performing after storage for up to 35 days at 37° C., (C) Best performing formulations are shown as time kinetics for up to 6 days at 25° C., (D) shows the infectivity of Ad5 in the best performing formulations after storage for up to 24 months at 5° C. The values show the mean±SD generated of at least 5 measurements per infected well.

FIG. 3: (A) shows the infectivity of Ad5 in liquid formulations (round 2) after different storage for up to 28 days at 37° C., (B) shows the infectivity of Ad5 in liquid formulations (round 2) after different storage for up to 12 days at 25° C., (C) shows the infectivity of Ad5 in liquid formulations (round 2) after different storage for up to 24 days at 5° C. The values show the mean±SD generated of at least 15 measurements (5 measurements from 3 biological replicates) per infected well.

The examples illustrate the invention:

### EXAMPLE 1: INITIAL EXCIPIENT SELECTION AND LIQUID STORAGE

### 1.1 Materials and Methods

### 1.1.1 Pre-Selected Formulations

For initial excipient selection, the formulation matrix, tailored for the Ad5 viral vectors, was composed of 40 formulations containing eight different amino acid combinations selected from Arg, Ala, Gly, Lys, His, Glu and Met in a concentration between 0.75 g/l and 31,235 g/l was used all formulation comprised 40 mg/ml saccharose, 2 mM MgCl₂ at a pH of 7.4. For comparison, original suppliers formulation (OF) 1,552 g/l Histidin, 50 g/l saccharose, 1 mM MgCl₂ at a pH of 7.4 and a positive control formulation comprising 2 mM MgCl₂ 60 g/l trehalose*2H2O, 0.05 g/l polysorbat 80 at a pH of 8 was used. An adenoviral stock solution (Ad5-CMV-EGFP: E1/E3-deleted human adenovirus, Serotype 5) stored at −80° C. with a concentration of 7.5×1010 IFU/ml in the original supplier formulation (OF), which was designed for frozen storage (Sirion Biotech GmbH; Germany) was employed.

### 1.1.2 Sample Preparation and Storage

Ad5 was diluted to 1×10⁸ IU/ml in original formulation (Sirion Biotech; Germany), or different amino acid based comprising different combinations and amounts of the eight different pre-selected amino acids in combination with sucrose, MgCl₂ according to 1.1.1.

The resulting Ad5 formulations were initially stored under short-term stress conditions at 37° C. for up to 35 days. In order to evaluate the predictive capability of the applied approach using a short-term storage model at 37° C. for liquid storage under real time conditions these samples were additionally stored for up to 6 months at 25° C. and for up to 24 months at 5° C. Infectivity of the Ad5 viruses was analyzed at day 0 and at different time point as indicated in FIGS. 1 and 2.

### 1.1.3 Infectivity Assay

In order to analyze the infective titer of the adenoviral vector formulations, antibody-based virus titration assays in adherent HEK 293 cell cultures were conducted. Antibody-mediated immunostaining of the adenoviral hexon protein was applied after successful amplification of the adenovirus in the infected cells. Therefore, 2.5×10⁵ HEK 293 cells in 500 μl per well were seeded in a 24-well plate and further used when cells started attaching to the surface (after 2-3 hours). Serial dilutions of the adenoviral samples were prepared and 50 μl of the resulting dilutions per well were used for infection of the cells. As for positive controls, aliquots of Ad5 in the original supplier formulation (Sirion Biotech, Germany) stored at −80° C. with a concentration of 1×10⁸ IU/ml were used. Cells were inoculated for 42±2 hours at 37° C., 5% CO₂ and subsequently fixed with methanol (Carl Roth GmbH & Co. KG; Germany). Immunostaining was done stepwise by incubation with the primary anti-Hexon protein antibody (Santa Cruz Biotechnology, Inc.; USA), the secondary horse radish peroxidase (HRP)-conjugated anti-mouse antibody (Cell Signaling Technology; USA) and an HRP enzymatic reaction with diaminobenzidine (Carl Roth GmbH & Co. KG; Germany). The number of infected cells was quantified by counting the stained (brown colored) cells under the light microscope. Each stained cell was considered as one infectious viral particle in order to calculate the Infective Units per milliliter (IU/ml) according to the standardized calculation procedure. The scope of detection allows titer determination between 9.87×10⁴ IU/ml and 2.04×10⁸ IU/ml.

### 1.1.4 Data Analysis

Data obtained for each time point during storage as provided in the Figures were analysed by DoE-based linear regression (R-Software; F-Statistics). For each amino acid F-values between −1, 0 and +1 indicate the linear influence of single amino acids on Ad5 stability and functionality calculated as Infective Unit per mL [IFU/ml] from hexon immunostaining. All experiments were done at least in triplicates and data are depicted as Mean±SD, except when indicated otherwise. Effects were considered statistically significant at p<0.1 (.), p<0.05 (*), p<0.01 (**), p<0.001 (***), respectively.

### 1.2 Results

After formulation of the original Ad5 viral vector preparation in these 40 formulations and in the original supplier formulation as a control, liquid storage under accelerated aging conditions at 37° C. was performed. Based on the first order regression of the infectivity results (FIG. 1A), the linear influences of each single amino acid used in the DoE calculation of the 40 formulations on Ad5 infectivity during liquid storage at 37° C. were evaluated (FIG. 1B). Either a positive, neutral or negative linear influence was determined for each single amino acid. The anti-oxidative effective amino acid methionine (AA8) demonstrated a significant (p<0.01) positive influence on the Ad5 stability after 14 days as well as 21 days short term liquid storage at 37° C. (FIG. 1B). The osmolytic amino acids alanine (AA2) and glutamine (AA7) also revealed a minor positive effect on Ad5 stability during liquid storage up to 21 days at 37° C. In contrast, the radical scavenging amino acid tryptophan (AA6) elicited a significant (p<0.01) negative influence up to 21 days short term liquid storage at 37° C. (FIG. 1B).

The identified linear effects of single amino acids were in line with formulations comprising these amino acids as determined in liquid storage experiments (37° C.) up to 21 days. The most effective stabilizing formulations (F1_3, F1_4, F1_10, F1_13, F1_16, F1_29, F1_39) partially retained Ad5 infectivity upon liquid storage at 37° C. for up to 21 days (see FIG. 2A) with a titer loss of approx. 1 to 2 log levels. In contrast, when the original supplier formulation was used, Ad5 infectivity was already lost after 14 days storage at 37° C. The composition of the

TABLE 2
Composition of most effective stabilizing formulations

| | | | L-Lysin | | | | | MgCl2 | |
|---|---|---|---|---|---|---|---|---|---|
| | | | mono- | | | | | *6H20 | |
| Formulation | Ala | Gly | HCl | His | Glu | Met | Saccharose | (2 mM ) | pH |
| No. | g/l | | | | | | | | |
| F1_3 | | | | 3 | 10 | 0.750 | 40 | 0.407 | 7.4 |
| F1_4 | | | 24.988 | 3 | | 1.500 | 40 | 0.407 | 7.4 |
| F1_10 | 10 | 10 | | 3 | 2 | 1.500 | 40 | 0.407 | 7.4 |
| F1_13 | 20 | | | 3 | 4 | | 40 | 0.407 | 7.4 |
| F1_16 | | 20 | | 3 | | 1.500 | 40 | 0.407 | 7.4 |
| F1_29 | 10 | | 31.235 | 3 | | 1.500 | 40 | 0.407 | 7.4 |
| F1_39 | 15 | | | 3 | | 0.750 | 40 | 0.407 | 7.4 |

After liquid storage for more than one month (5 weeks) at 37° C., only Ad5 in formulation F1_29 remained active with a titer loss of approx. 3 log levels while no infectivity (limit of detection (LOD) reached) could be found with all other formulations. All stabilizing formulations contained either 2 or 3 selected amino acids with positive linear influence (FIG. 1) and tryptophan (AA6) which was shown earlier to elicit significant negative influence on Ad5 stability.

In contrast to the best stabilizing formulations such as F1_29, the weakest stabilizing formulations only maintained Ad5 infectivity up to 14 days storage at 37° C. (see FIG. 2B). In line with the results of the linear regression analysis of the DoE-based infectivity data these weakly stabilizing formulations contained amino acid tryptophan (AA6). The best performing formulations did not comprise tryptophan (AA6).

Based on these results, the most effective stabilizing excipients methionine (AA8), alanine (AA2) and glutamine (AA7) as well as the elimination of tryptophan (AA6) were considered for long-term storage experiments and further iterative optimization of the formulations.

The linear regression of the DoE based Ad5 infectivity results analyzed at indicated time points, according to guideline ICH Q1, during liquid storage at 25° C. (up to 12 months) and 5° C. (up to 24 months) revealed similar influences of single amino acids on the Ad5 stability during storage at both temperatures compared to liquid storage at 37° C.). For example, similar to the results regarding the linear influences of the single amino acids during short-term storage of Ad5 at 37° C. (FIG. 1B), the amino acid methionine (AA8) also significantly stabilized Ad5 during liquid storage at 25° C. (FIG. 10) and at 5° C. (FIG. 1D). In contrast, amino acid tryptophan (AA6) elicited significant negative effects on Ad5 stability during liquid storage at all temperatures (FIGS. 1B-D).

Results of the seven best-of stabilizing at 37 C liquid formulations (F1_3, F1_4, F1_10, F1_13, F1_16, F1_29, F1_39) are shown in FIG. 2A. FIG. 2C depicts the results obtained at indicated time points during liquid storage for up to 6 months at 25° C. These formulations are similar to the best-of formulations during liquid storage under accelerated aging conditions at 37° C. The loss of only maximal 1 log level of the virus infectivity was observed after 3 months and 1-2 log after 6 months storage at 25° C. (FIG. 2C). In contrast, during storage for 3 months the Ad5 virus formulated in the original supplier formulation (OF) completely lost infectivity.

As shown in FIG. 2D, the stabilizing effects of the seven best-of stabilizing formulations (F1_3, F1_4, F1_10, F1_13, F1_16, F1_29, F1_39) and their impact on Ad5 infectivity during liquid storage at 5° C. are shown over time up to 24 months. A dramatic loss of infectivity was found for Ad5 in OF already after three months storage at 5° C. (FIG. 2D). Accordingly, after 24 months storage at 5 C a complete loss of the Ad5 infectivity in the OF was observed. All stabilizing formulations almost completely maintained the Ad5 viral infectivity during long-term storage at 5° C. for up to 24 months. These results are in line with the calculation of the linear influences of single amino acids on the Ad5 stability. For example, the formulations F1_3, F1_4, F1_10, F1_13, F1_16, F1_29, F1_ 39 lack acid tryptophan (AA6) that was shown to have negative linear effects at 37° C. (see FIG. 1). Formulations without methionine (AA8) (e.g. F1_14, F1_15, F1_25, F1_26, F1_30, F1_31, F1_33) resulted in a total loss after 24 months at 5° C. or to a loss of infectivity up to 1 to 2 log levels during liquid storage for 24 months at 5° C. (FIG. 2D). During long-term liquid storage for up to 6 months at 25° C. the same formulations showed only minor stabilizing effects on functional integrity of the viral vector.

Interestingly, the most effective stabilizing formulations (F1_20, F1_22, F1_34, F1_36) after 24 months liquid storage at 5° C. all contained amino acid tryptophan, except formulation 34, but also contained methionine (AA8), suggesting a partially masking of the negative influence of acid tryptophan (AA6) by the stabilizing effect of methionine (AA8) during long term liquid storage. In contrast, during liquid storage under accelerated aging conditions the negative influence of acid tryptophan (AA6) is more pronounced also in combination with methionine (AA8).

The DoE-based overall evaluated linear influences of single amino acids on the Ad5 stability during liquid storage were found likely to be superimposed by the negative or positive effects of specific excipients in the particular formulations during liquid storage at different temperatures. For example, poor stabilizing formulations (F1_5, F1_7, F1_17, F1_22, F1_25, F1_28, F1_31, F1_34; see FIG. 1A) containing high amounts of AA1 were associated with negative effects on Ad5 stability during short-term storage at 37° C. However, as shown in FIG. 1, the evaluation of the linear influence of single amino acids revealed a neutral influence of AA1. The rather poor stabilizing effects of formulations 22 and 34 during short term storage at 37° C. associated with AA1 may be overcome by the stabilizing effects of methionine (AA8) during long-term storage. Moreover, the previously evaluated significant negative influence of the aromatic amino acid tryptophan (AA6) reflected in the poor stabilizing effects of formulations F1_14, F1_15, F1_25, F1_30, F1_31, F1_33 was completely abolished during long-term storage in formulations F1_20, F1_22 and F1_36 that all contained AA6 in combination with tryptophan (AA8).

### EXAMPLE 2: OPTIMIZATION OF THE STABILIZING AD5 FORMULATIONS

### 2.1 Materials and Methods

Based on formulations F1-16 and F1-29 the formulations were modified as shown in Table 3.

TABLE 3
Modified formulations

| | | | | | L- | | | | | | MgCl2 | |
|---|---|---|---|---|---|---|---|---|---|---|---|---|
| | | | | | Lysin | | | | | | *6H20 | |
| | | | | | mono- | | | | | | (2 mM | |
| Formulation | | Arg | Ala | Gly | HCl | His | Glu | Met | Saccharose | Mannitol | fix) | pH |
| No | | g/l | | | | | | | | | | |
| F1_29 | F2_1 | | 10 | | 31.235 | 3 | 0 | 1.500 | 40 | | 0.407 | 7.4 |
| | F2_2 | | | 8.426 | 31.235 | 3 | 0 | 1.500 | 40 | | 0.407 | 7.4 |
| | F2_3 | 29.804 | 10 | | | 3 | 0 | 1.500 | 40 | | 0.407 | 7.4 |
| | F2_4 | | 10 | | 31.235 | 3 | 0 | 1.500 | 40 | | | 7.4 |
| | F2_5 | | 10 | | 31.235 | 3 | 0 | 1.500 | | 21.290 | 0.407 | 7.4 |
| | F2_6 | | 10 | | 31.235 | 3 | 4 | 1.500 | 40 | | 0.407 | 7.4 |
| F1_13 | F2_7 | | 20 | | | 3 | 4 | | 40 | | 0.407 | 7.4 |
| | F2_8 | | 20 | | | 3 | 4 | | 40 | | | 7.4 |
| | F2_9 | | 20 | | | 3 | 4 | 1.500 | 40 | | 0.407 | 7.4 |

The modified formulations were stored for 37° C. for up to 28 days, 25° C. for up to 12 months and 5° C. for up to 24 months. Samples were analysed as described for Example 1 at the time points indicated in FIG. 3.

### 2.2 Results

The identified most effective stabilizing amino acids methionine (AA8), alanine (AA2) and glutamine (AA7) and two of the most effective stabilizing amino acid compositions (FIG. 3A; formulations F1_13 and F1_29) of the first round were the basis for the second stabilization round for the Ad5 vector. Although formulation F1_13 did not contain methionine (AA8), this formulation was one of the most effective Ad5 stabilizing formulations in the DoE round (FIG. 3A). It was assumed that formulations containing high amounts of the osmolytic amino acid alanine (AA2) and intermediate amounts of the osmolytic acidic amino acid glutamine (AA7) as well as sucrose and MgCl₂ may overcome the lack of the anti-oxidative amino acid methionine (AA8). Formulation F1_13 and F1_29 were modified accordingly. Modified formulations are shown in Table 3.

Surprisingly, in both of the modified initial formulations, omission of MgCl₂ resulted in an increased stability under long term storing conditions at 25° C. as shown in FIG. 3B (F2_1 vs F2_4 and F2_7 vs F2_8.

In comparison to long term storing conditions at 25° C. (FIG. 38) the effect of the omission of MgCl₂ was less pronounced at short term storage at 37° C. (FIG. 3A) or storage at 5° C. (FIG. 3A).

Liquid storage for 6 months at 25° C. led to a loss of titer only between 1.5 and 2 log levels of the Ad5 infectivity for most formulations. Ad5 viral vector formulated in the formulations F2_1; F2_2; F2_4; F2_5; F2_6; F2_8 and F2_9 (see; see Table 3) retained the infective titer even after 9 months storage at 25° C. The best stabilizing effect were observed with the formulation F2_4 w/o MgCl₂, which performed best with a titer of 1.23×10⁵ IFU/ml after 12 months at 25° C. (FIG. 38).

These results suggest that in contrast to the teachings of the prior art, which rely on the presence of MgCl₂ for stabilizing viral vectors, stabilization of viral vectors may be improved in solutions which are free or substantially free of Mg²⁺ and/or salts thereof, and preferably free or substantially free of divalent cations.

1. A method for preparing a composition comprising a viral vector, the method comprising the steps:
(a) providing viral vectors;
(b) providing a solution comprising at least one sugar and at least three different excipients selected from hydrophilic and amphiphilic excipients, wherein the excipients are characterized by polar, aliphatic, aromatic, negatively charged, and/or positively charged functional groups, and
wherein preferably the at least three different excipients comprise or are amino acids; and
wherein the solution is free or substantially free of Mg²⁺ and/or salts thereof;
(c) mixing the viral vectors of step (a) with the solution of step (b).

2. The method of claim 1, wherein the solution is free or substantially free of Ca²⁺, Mn²⁺, Cu²⁺, Zn²⁺, and or Ni²⁺ and/or salts thereof, or wherein the solution is free or substantially free of any divalent cations.

3. The method of claim 1 or 2, wherein the at least three amino acids, at least provide one anti-oxidative functional group and at least one osmolytic function and at least one buffering function and at least one charged functional group.

4. The method of any of the preceding claims further comprising the step (d) of storing the composition obtained by mixing the viral vectors of step (a) with the solution of step (b) in liquid state.

5. The method of claim 4, wherein the composition is stored in liquid state in step (d) for at least 30 days, more preferably at least 6 months, even more preferably 9 months and most preferably at least 12 months.

6. The method of claims 4 and 5, wherein the composition is stored in liquid state in step (d) at a temperature between 4° and 30°, preferably between 20° C. and 27° C.

7. The method of claims 4 to 6, wherein the composition storage in liquid state in step (d) for 6 months at 25° C. leads to a loss of infectivity titer of no more than between 1.5 and no more than 2 log levels.

8. The method of claims 4 to 6, wherein the composition storage in liquid state in step (d) for over 12 months at 25° C. in step (d) of the method of the invention, the loss in infectivity titer of the viral vector comprised in the composition may be no more than 3 log levels.

9. The method of any of the preceding claims, wherein the viral vector is selected from the group consisting of adenovirus, Adenovirus-associated virus (AAV), lentivirus, vesicular stomatitis virus (VSV), MVA, or herpesviruses.

10. The method of any of the preceding claims, wherein the viral vector is a virus like particle.

11. The method of any of the preceding claims, wherein the viral vectors of (a) are viral vectors that have been reconstituted immediately after harvesting from cell cultures and purification.

12. The method of any of the preceding claims, wherein the viral vector-based particles present in the composition have a particle size distribution with a polydispersity index (PDI) of less than 0.5.

13. A composition obtained or obtainable by the method according to any of the preceding claims.

14. A composition comprising a viral vector and a solution according to claim 1.

15. The composition according to claim 13 or 14, wherein the composition is characterized by a loss of infectivity titer of no more than between 1.5 and 2 log levels upon storage for 6 months at 25° C. and/or by a loss of infectivity titer of no more than 3 log levels upon storage of the composition over 12 months at 25° C.
